(19) United States
(12) Patent Application Publication
(10) Pub. No.: US 2010/0021940 A1
(43) Pub. Date: Jan. 28, 2010

(54) Method for Determining the Activity of a Protease in a Sample

(75) Inventors: Anders Blomgren, Lund (SE); Anders Eriksson, Lund (SE); Thomas Hansson, Lund (SE); Keith Jolley, loughborough (GB); Matti Lepisto, Lund (SE); Karin Von Wachenfeldt, Lund (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/296,568

(22) PCT Filed: Apr. 11, 2007
(86) PCT No.: PCT/SE2007/000339

§ 371 Date: Sep. 14, 2009

Related U.S. Application Data
(60) Provisional application No. 60/791,512, filed on Apr. 12, 2006.

(51) Int. Cl. C12Q 1/37; G01N 33/573; C07K 2/00; C07C 205/00

(52) U.S. Cl. 435/7.8; 435/23; 530/300; 564/463

(57) ABSTRACT

There is provided a method for determining the activity of a protease in a sample. The method comprises (i) admixing said sample with a substrate, wherein the substrate has the formula (1a) wherein: R₁ is a hydrocarbyl group; R₂ is a first peptide moiety; R₃ is a second peptide moiety and X is selected from the group consisting of O, S and NH; Y₁ is a suitable substituent; Y₂ is a suitable substituent; and (ii) determining the activity of said protease by detecting the presence of a reporter having the formula H—X—R₁, wherein: X is selected from the group consisting of O, S and NH; R₁ is a hydrocarbyl group. The substrate and reporter are useful for determining the efficacy of protease-modulators and candidate protease-modulators and in the diagnosis of a disease or disorder in a subject.

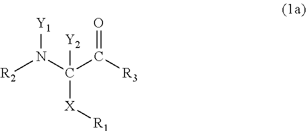

### FIELD

The present invention relates to a method, as well as to a substrate for use in said method and a reporter generated by said method.

In particular, the present invention relates to methods for determining the activity of a protease in a sample comprising admixing said sample with a substrate and determining the activity of said protease by detecting the presence of a reporter.

The present invention also relates to a substrate and a reporter useful in a method for determining the activity of a protease in a sample comprising admixing said sample with a substrate and determining the activity of said protease by detecting the presence of a reporter.

In another aspect the present invention relates to the use of the substrate and reporter for determining the efficacy of protease-modulators and candidate protease-modulators.

In a further aspect the present invention relates to the use of the substrate and reporter in the diagnosis of a disease or disorder in a subject.

### BACKGROUND

Proteases, such as metalloproteinases, are capable of cleaving a broad range of substrates such as collagen, proteoglycan and fibronectin. Hence proteases, such as metalloproteinases, are considered to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Examples of metalloproteinases include the matrix metalloproteinases (MMPs)—such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family—which includes the secretases and sheddases such as TNF converting enzymes (ADAM10 and TACE); the astacin family—which includes enzymes such as procollagen processing proteinase (PCP); and other metalloproteinases such as aggrecanase, the endothelin converting enzyme family and the angiotensin converting enzyme family.

To date several assays have been used to determine the presence of proteases, such as metalloproteinases, in samples.

For example, Hanemaaijer et al (1999, Ann. N.Y. Acad. Sci., vol 878, 141-149) disclose an assay to determine the activity of MMP9 in the urine of patients with tumours. This assay requires the capture of MMP9 from biological fluids using MMP9 specific antibodies followed by a wash step and then incubation with a modified urokinase as a substrate and a chromogenic substrate.

Bicket et al (1993, Analytical chemistry 212:58-64) disclose a fluorogenic peptide substrate (Dnp-Pro-Cha-Gly-Cys(Me)-His-Ala-Lys-(Nma)-NH₂) which is hydrolysed by MMP1 and MMP9. In order to determine the activity of the enzymes, the assay uses the fluorogenic peptide substrate and purified MMP9 or purified MMP1.

Mucha et al (1998, J Biological chemistry 273:2763-2768) disclose synthetic substrates which are cleaved by MT1-MMP and ST3. The assays described in Mucha et al teach the use of purified catalytic domains of ST3 and MT1-MMP and fluorescent labelled substrates.

Tung et al (1999-Bioconjugate Chem 10:892-896) disclose the use of a near-infrared fluorescence (NIRF) probe with a CaD substrate spacer to determine cathepsin D activity in a cell culture.

The fluorogenic substrate Dabcyl-Gaba-Arg-Pro-Lys-Pro-Val-Glu-Nva-Trp-Arg-Glu(EDANS)-Gly-Lys-NH₂ (TNO003) was used to monitor stromelysin (MMP-3) activity in the synovial fluid from patients with rheumatoid arthritis (B. Beekman et al, FEBS Letters 1997, 418(3), 305-309).

WO-A-03/025125 discloses polypeptide sequences which are specific for MMP2, MMP9 and MT1-MMP (MMP14). These peptide sequences may be linked to therapeutic moieties or to diagnostic agents such as fluorophores.

WO-A-03/102544 discloses polypeptide sequences which are specific for MMP13 and the attachment of fluorescent, calorimetric, radioactive and luminescent labels to said polypeptide sequences.

A problem with the prior art assays is that the substrates require labelling with, for example, fluorophores or radioactive labels. Another problem with the prior art assays is that cleavage at any peptide bond is detected—cleavage at a specific peptide bond cannot be detected. A further problem with some of the prior art assays is that the protease first needs to be purified before the activity of said protease can be detected. In addition, the prior art assays cannot be used to determine the activity of protease modulators.

The present invention seeks to address/alleviate some of the problems associated with the prior art.

### BROAD ASPECTS

In the following text, reference is made to substrate compounds of the general formulae (1a), (1b), (1c) and (1d). For ease of reference, and wherever applicable, these compounds are collectively referred to as substrate compounds of the general formula (1). Thus, a reference to substrate compounds of the general formula (1) applies equally to substrate compounds of the general formulae (1a), (1b), (1c) and (1d). In addition, it is to be noted that preferred aspects for compounds of the general formula (1), (1a), (1b), (1c) or (1d) apply equally to any of the other of compounds of the general formula (1), (1a), (1b), (1c) or (1d).

In one broad aspect of the present invention there is provided a method for determining the activity of a protease in a sample comprising the steps of:
(i) admixing said sample with a substrate, wherein the substrate has the formula (1a)

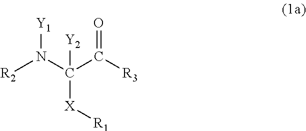

wherein:
   R₁ is a hydrocarbyl group
   R₂ is a first peptide moiety
   R₃ is a second peptide moiety and
   X is selected from the group consisting of O, S and NH;
   Y₁ is a suitable substituent; and
   Y₂ is a suitable substituent;
      and
(ii) determining the activity of said protease by detecting the presence of a reporter having the formula H—X—R₁ wherein:
   X is selected from the group consisting of O, S and NH; and
   R₁ is a hydrocarbyl group.

Determining the activity of a protease in a sample means a qualitative assessment and/or a quantitative assessment of protease activity in the sample.

Preferably Y₁ is H.

Preferably Y₂ is H.

Thus, in a highly preferred aspect of the present invention the substrate has the formula (1b):

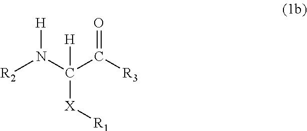

wherein:
   R₁ is a hydrocarbyl group
   R₂ is a first peptide moiety
   R₃ is a second peptide moiety and
   X is selected from the group consisting of O, S and NH.

Preferably R₁ is a mono or polycyclic ring structure.

Preferably R₁ comprises 1, 2 or 3 hydrocarbyl rings. In some instances, more preferably R₁ comprises 2 or 3 hydrocarbyl rings.

If R₁ comprises 2 hydrocarbyl rings, preferably those rings are unfused rings.

If R₁ comprises 3 hydrocarbyl rings, preferably 2 of those rings are fused rings.

One or more of the hydrocarbyl rings may contain heteroatoms—such as one or more N.

One or more of the hydrocarbyl rings may be substituted.

One or more of the hydrocarbyl rings may be unsaturated.

Preferably R₂ comprises at least two amino acid groups, wherein said groups are natural amino acids or unnatural amino acids or combinations thereof.

Preferably R₂ comprises at least three amino acid groups, wherein said groups are natural amino acids or unnatural amino acids or combinations thereof.

Preferably R₃ comprises at least two amino acid groups, wherein said groups are natural amino acids or unnatural amino acids or combinations thereof.

For some embodiments, preferably R₃ comprises at least three amino acid groups, wherein said groups are natural amino acids or unnatural amino acids or combinations thereof.

Thus, in a preferred aspect of the present invention the substrate has the formula (1c):

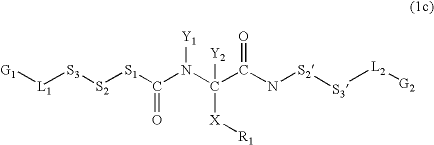

wherein
R₁ is a hydrocarbyl group
each of S₁, S₂, S₃, S₂′ and S₃′ is independently selected from a natural amino acid or an unnatural amino acid;
L₁ is a bond or one or several amino acid(s);
L₂ is a bond or one or several amino acid(s);
G₁ is a suitable end group;
G₂ is a suitable end group;
X is O, S or NH;
Y₁ is preferably H; and
Y₂ is preferably H.

By way of example, L₁ may be one or more α, β, . . . etc. . . . ω-amino acids—such as (by way of example) Gly-Gly-Gly or γ-butyramide.

By way of example, L₂ may be one or more α, β, . . . etc. . . . ω-amino acids—such as (by way of example) Gly-Gly-Gly or γ-butyramide.

By way of example, G₁ may be an end group, a protecting group, a chromophoric group or a fluorophoric group.

By way of example, G₂ may be an end group, a protecting group, a chromophoric group or a fluorophoric group.

In a preferred aspect of the present invention the substrate has the formula (1d):

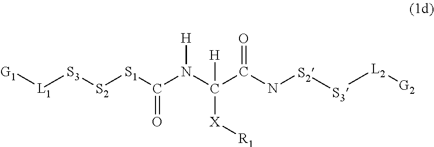

wherein
each of S₁, S₂, S₃, S₂′ and S₃′ is independently selected from a natural amino acid or an unnatural amino acid;
L₁ is a bond or one or several amino acid(s);
L₂ is a bond or one or several amino acid(s);
G₁ is a suitable end group;
G₂ is a suitable end group;
X is O, S or NH; and
R₁ is selected from panel 1:

### Panel 1:

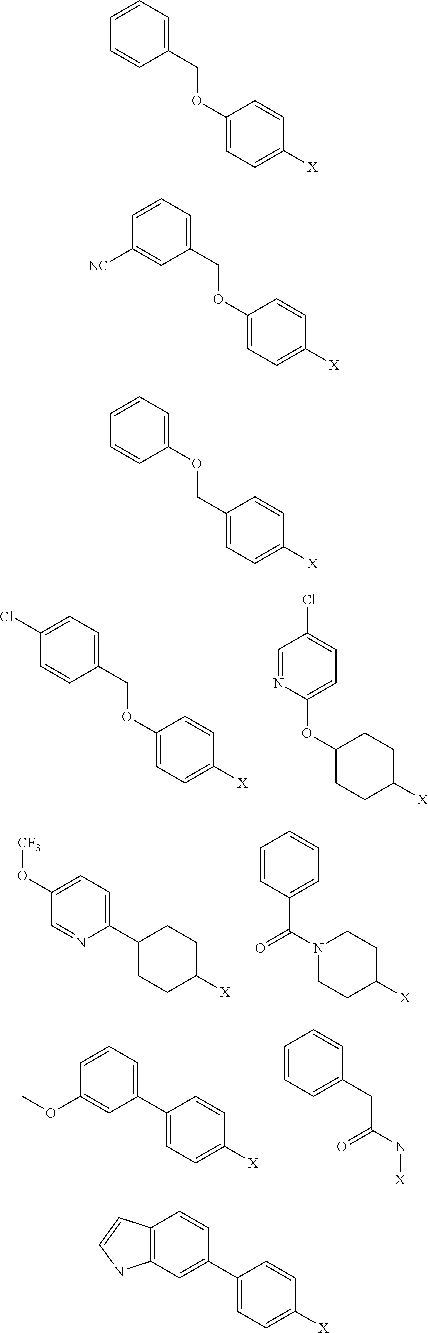

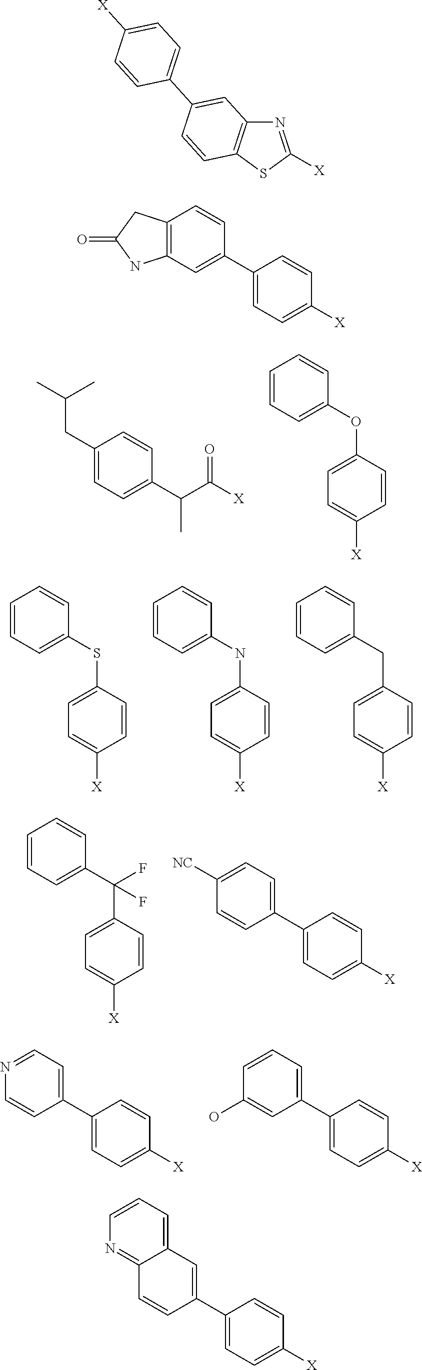

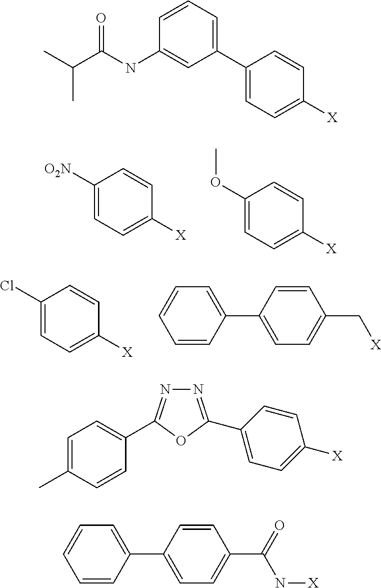

wherein X in panel 1 denotes the remainder of the substrate of formula (1d).

### FURTHER ASPECTS

In one aspect of the present invention there is provided a method for determining the efficacy of a protease-modulator; wherein said method comprises the steps of:

(i) admixing said protease-modulator with a protease and a substrate having the formula (1) and
(ii) determining the activity of said protease by detecting the presence of a reporter having the formula H—X—R₁ as defined herein.

After this method, more of the protease-modulator may be prepared and/or then formulated. The formulating step may include one or more of: derivatisation of the protease-modulator; forming the protease-modulator into a pro-drug; admixing the protease-modulator with one or more pharmaceutically acceptable carrier(s), diluent(s) or excipient(s).

In another aspect of the present invention there is provided a method for determining the efficacy of a candidate protease-modulator; wherein said method comprises the steps of:

(i) admixing said candidate protease-modulator with a protease and a substrate having the formula (1) and
(ii) determining the activity of said protease by detecting the presence of a reporter having the formula H—X—R₁ as defined herein.

After this method, more of the candidate protease-modulator may be prepared and/or then formulated. The formulating step may include one or more of: derivatisation of the protease-modulator; forming the protease-modulator into a pro-drug; admixing the protease-modulator with one or more pharmaceutically acceptable carrier(s), diluent(s) or excipient(s).

In another aspect of the present invention there is provided a method for identifying a protease-modulator; wherein said method comprises the steps of:

(i) admixing a candidate protease-modulator with a protease and a substrate having the formula (1) and
(ii) determining the activity of said protease by detecting the presence of a reporter having the formula H—X—R₁, as defined herein.

In another aspect of the present invention there is provided a process comprising the steps of identifying a protease-modulator, preparing more of an identified protease-modulator and/or then formulating more of the identified protease-modulator; wherein said identification part comprises the steps of:

(i) admixing a candidate protease-modulator with a protease and a substrate having the formula (1) and
(ii) determining the activity of said protease by detecting the presence of a reporter having the formula H—X—R₁, as defined herein.

The formulating step may include one or more of: derivatisation of the protease-modulator; forming the protease-modulator into a pro-drug; admixing the protease-modulator with one or more pharmaceutically acceptable carrier(s), diluent(s) or excipient(s).

In a further aspect, the present invention provides a substrate having the formula (1).

In another aspect, the present invention provides a substrate capable of being cleaved at a peptide bond between a carbonyl group and a —NH—CH(X—R₁)-group by a protease; wherein R₁ is a hydrocarbyl group and X is selected from the group consisting of O, S and NH.

The present invention provides, in a further aspect, a reporter having the formula H—X—R₁, wherein R₁ is a hydrocarbyl group and X is selected from the group consisting of O, S and NH; and wherein said reporter is derived from a substrate of the formula (1).

In another aspect there is provided a reporter having the formula H—X—R₁, wherein R₁ is a hydrocarbyl group and X is selected from the group consisting of O, S and NH; and wherein R₁ is selected from panel 1:

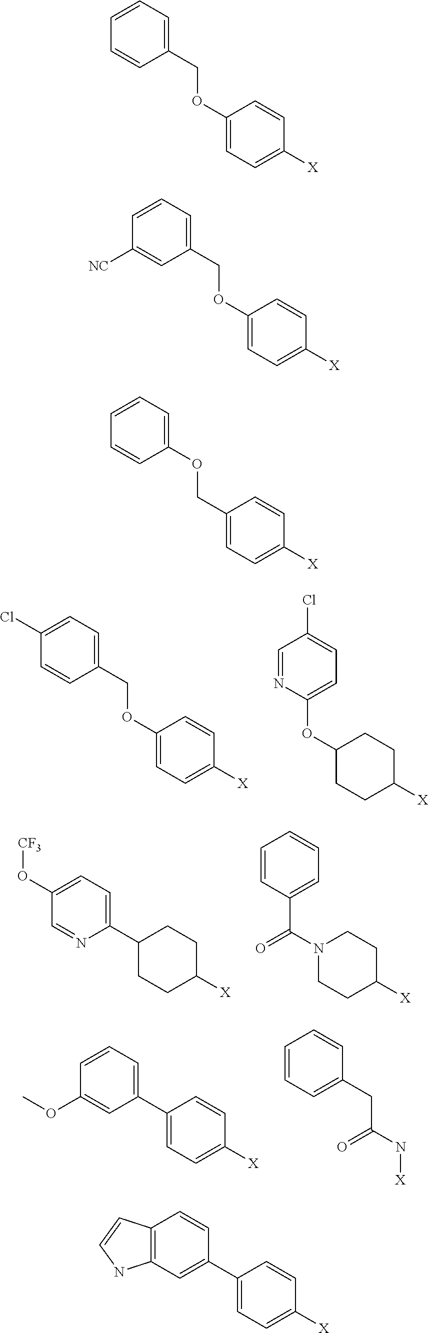

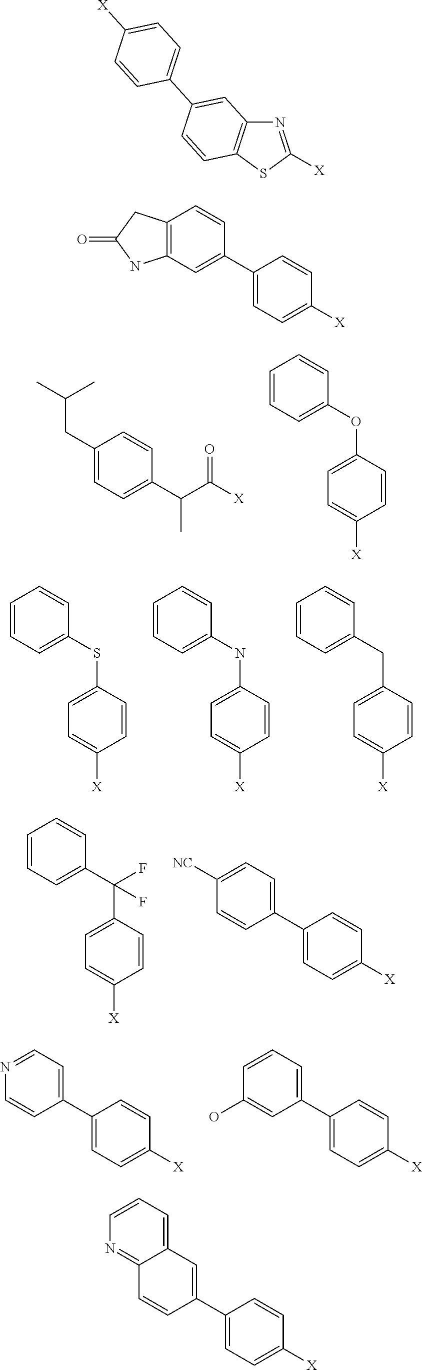

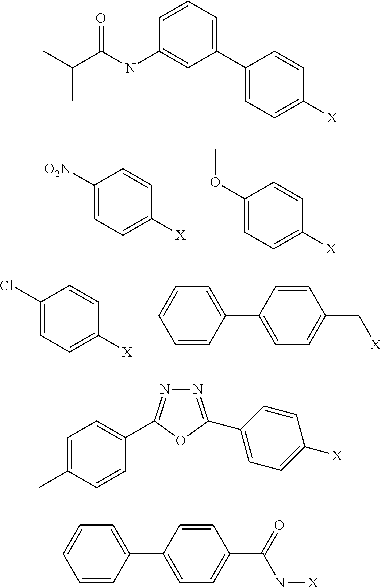

### (Panel 1)

wherein X in panel 1 denotes the remainder of the reporter.

A further aspect of the present invention provides a kit for determining the activity of a protease in a sample comprising:

(i) a substrate having the formula (1); and
(ii) means for detecting a reporter having the formula H—X—R₁, as defined herein in the sample.

In another aspect, the present invention provides the use of a substrate having the formula (1) for detecting protease activity in a sample.

The present invention provides, in a further aspect, the use of a reporter having the formula H—X—R₁ as defined herein for detecting protease activity in a sample.

The present invention provides, in another aspect, a method for diagnosing a disease or a disorder in a subject comprising the steps of obtaining a sample from said subject and determining the activity of a protease in said sample by a method comprising the steps of:

(i) admixing said sample with a substrate having the formula (1);
(ii) determining the activity of said protease by detecting the presence of a reporter having the formula H—X—R₁, as defined herein.

In a further aspect, the present invention provides a substrate having the formula (1) for use in the diagnosis of a disease or disorder in a subject.

In another aspect, the present invention provides a reporter having the formula H—X—R₁, as defined herein for use in the diagnosis of a disease or disorder in a subject.

In another aspect there is provided a method for determining the activity of a protease in a sample comprising admixing said sample with a substrate wherein said substrate is capable of being cleaved at a peptide bond between a carbonyl group and a —NH—CH(X—R₁)-group by said protease; and determining the activity of said protease by detecting the presence of a reporter having the formula H—X—R₁. X and R₁ are as defined herein.

In a further aspect there is provided a method for determining the efficacy of a protease-modulator comprising treating a sample with a protease-modulator and/or obtaining a sample from a subject treated with a protease-modulator; admixing said sample with a substrate wherein said substrate is capable of being cleaved at a peptide bond between a carbonyl group and a —NH—CH(X—R₁)-group by said protease; and determining the activity of said protease by detecting the presence of a reporter having the formula H—X—R₁. X and R₁ are as defined herein.

There is provided, in another aspect, a method for determining the efficacy of a candidate protease-modulator comprising treating a sample with a candidate protease-modulator and/or obtaining a sample from a subject treated with a candidate protease-modulator; admixing said sample with a substrate wherein said substrate is capable of being cleaved at a peptide bond between a carbonyl group and a —NH—CH(X—R₁)— group by said protease; and determining the activity of said protease by detecting the presence of a reporter having the formula H—X—R₁. X and R₁ are as defined herein.

### ADVANTAGES

The methods as described herein detect the activity of a protease in a sample and not simply the levels of mRNA encoding the protease or levels of the polypeptide itself in the sample.

Surprisingly the substrate according to the present invention obviates the requirement for a fluorescent, a luminescent, a calorimetric or a radioactive label to be added to the substrate in order for protease activity to be determined.

Surprisingly, the assay methods described herein measure cleavage at a bond of interest. In the present invention, when a protease cleaves the substrate at the bond of interest it results in the production (i.e. release) of the reporter. If a protease cleaves the substrate elsewhere then the reporter is not produced.

The specificity profile of any given protease is largely determined by the substrate amino acid sequence extending in both directions from the cleavage site. The amino acids adjacent to either side of the cleaved bond are frequently the most important determinants for specificity, although important determinate amino acids can be as far as 6 positions away from the cleavage site. (J. D. A. Tyndell et al, 2005, Chem. Rev. 105, 973-999—a review of substrate recognition by proteases). Accordingly, the potential for cleavage at a specific site can be expected to increase with the length of the substrate with an appropriate sequence, at least to a limit when secondary structural effects (i.e. folding, helical conformations) start to be important. However, without wishing to be bound by theory, as the length of the substrate increases there is an increased risk that the longer sequence contains cleavage sites for other proteases between the cleavage site(s) of a protease(s) of specific interest. Thus, the ability to detect a specific bond cleavage as described herein advantageously results in assay that can determine cleavage by a specific protease.

The methods as described herein permit other enzymes to be present in the sample.

The methods as described herein can be used to monitor the effect of protease-modulators because the methods do not require the use of a wash step.

The assay methods described herein do not require the use of antibodies which are specific for proteases.

The methods described herein can use tissues as the sample.

The substrates as described herein are stable, in the absence of proteases of specific interest, in the sample such as blood serum and plasma.

### PREFERRED ASPECTS

Preferably the protease-modulator is a protease-inhibitor.

Preferably the protease-modulator is a protease-activator. An example of a protease-activator is zymosan.

Other examples of protease-activators can be found in Rivera-Marrero et al (2002, Am J Physiol Lung Cell Mol Physiol 282: L₅₄₆-L₅₅₅); Kadish et al (1986, Immunol. Res. 5:129); Czop et al (1978, J. Immunol. 120:1132); Williams (1996, Clin. Immunother. 5:392); Ross et al (1999, Immunopharmacology, 42:61); Williams et al (1978, J Reticuloendothel. Soc. 23:479); Kokoshis et al (1978 Science 199:1340); Itoh (1997, Mediat. Inflamm. 6:267); Williams (1997, Mediat. Inflamm. 6:247).

Preferably the candidate protease-modulator is a candidate protease-inhibitor.

Preferably, for other embodiments, the candidate protease-modulator is a candidate protease-activator.

Preferably the sample is selected from the group consisting of any mammalian biofluid such as: urine, whole blood, blood plasma, blood serum, synovial fluid, saliva, sputum, bronchoalveolar fluids, cerebrospinal fluid, nasal lavage, lung lining fluid, tear fluid and skin blister fluids.

Preferably the sample is selected from the group consisting of: a cell culture, tissue, tissue slices and homogenised tissue.

Preferably the protease is a mammalian matrix metalloproteinase (MMP) EC3.4.24-.

Preferably the matrix metalloproteinase is selected from the group consisting of MMP1 (EC3.4.24.7), MMP2 (EC3.4.24.24), MMP3 (EC3.4.24.17), MMP8 (EC3.4.24.34), MMP9 (EC3.4.24.35), MMP12 (EC3.4.24.65) and MMP13 (EC3.4.24-). More preferably said matrix metalloproteinase is MMP9 (EC3.4.24.35). In an alternative more preferable embodiment said matrix metalloprotein is MMP12 (EC3.4.24.65). In yet another alternative preferable embodiment said matrix metalloprotein is MMP13 (EC3.4.24-).

Preferably the hydrocarbyl group R₁ is selected from the group consisting of an aryl, heteroaryl, aryloxyaryl, biaryl, alkyl, cycloalkyl, heterocycloalkyl group and derivatives thereof.

More preferably R₁ is selected from the group consisting of an aryl, heteroaryl, aryloxyaryl, biaryl and derivatives thereof.

Preferably R₁ comprises from 10 to 25 carbon atoms, preferably from 10 to 20 carbon atoms.

In a highly preferred embodiment R₁ is selected from panel 1; wherein X in panel 1 denotes the remainder of the substrate.

In a highly preferred embodiment R₁ is selected from panel 1; wherein X in panel 1 denotes the remainder of the reporter.

Preferably the substrate is methyl 1-acetyl-L-prolyl-L-leucylglycyl-α-R-(4-nitrophenylamino)-glycyl-L-leucyl-β-alaninate.

Preferably the reporter is 4-nitroaniline, biphenyl-4-yl-methanol, 4-(5-p-tolyl-[1,3,4]oxadiazol-2-yl)-phenylamine, N-hydroxy-2-phenyl-acetamide, biphenyl-4-carboxylic acid hydroxyamide, or 2-(-4-Isobutyl-phenyl)-propionic acid (Ibuprofen®).

More preferably the reporter having formula H—X—R₁ is 4-nitroaniline, 4-(5-p-Tolyl-[1,3,4]oxadiazol-2-yl)-phenylamine, or biphenyl-4-yl-methanol.

In a highly preferred embodiment the reporter is 4-nitroaniline.

Preferably the disease or disorder is selected from a group consisting of: rheumatoid arthritis, osteoarthritis; multiple sclerosis; airway diseases such as asthma, rhinitis, chronic bronchitis, chronic obstructive bronchioliti, airway fibrosis and chronic obstructive pulmonary disease (COPD)

More preferably the disease or disorder is osteoarthritis, asthma or chronic obstructive pulmonary disease (COPD).

In one highly preferred embodiment the disease or disorder is osteoarthritis.

In another highly preferred embodiment the disease or disorder is chronic obstructive pulmonary disease (COPD).

### DETAILED DESCRIPTION

### Protease

The term “protease” as used herein refers to an enzyme which hydrolyses peptide bonds. In other words, proteases are enzymes which break peptide bonds between amino acids of proteins—this process is referred to as proteolytic cleavage. Proteases are also referred to as proteinases, peptidases or proteolytic enzymes.

Proteases are classified under the Enzyme nomenclature number EC 3.4.*. Proteases are classified on the basis of the most prominent functional group in their active site. At present there are 6 classes of proteases: serine proteases, cysteine proteases, aspartic acid proteases, threonine proteases, glutamic acid proteases and metalloproteinases.

A database of known proteases is available at http://merops.sanger.ac.uk (Rawlings, N. D., Tolle, D. P. & Barrett, A. J. (2004) MEROPS: the peptidase database. Nucleic Acids Res. 32 Database issue, D160-D164).

### Metalloproteinases

Metalloproteinases (or metalloproteases) bind a metal ion such as Zn²⁺ or Ca²⁺ in their active site. The ion usually serves to co-ordinate two to four side chains and it is indispensable for the activity of the enzyme. The ion itself is also coordinated by a water molecule, which is also crucial for catalytic activity.

Metalloproteinases are secreted as pro-enzymes and activation requires cleavage of a pro-peptide sequence bound to the active site zinc atom. This activity can be stimulated by incubating whole blood with zymosan.

There are two subgroups of metalloproteinases: metallocarboxipeptidases (EC 3.4.17) and matrix metalloproteinases (MMP, also referred to as metalloendopeptidases or matrixins-EC: 3.4.24).

Preferably the protease is a matrix metalloproteinase (MMP) EC 3.4.24.-.

### Matrix Metalloproteinases (MMPs)-EC 3.4.24.-

MMPs are extracellular proteases that function at a neutral pH to cleave a wide variety of substrates (such as basement membrane and extracellular matrix components, growth and death factors, cytokines, and cell and matrix adhesion molecules—Bergers and. Coussens Curr. Opin. Genet. Dev. 2000 10 120; Visse and Nagase Circ. Res. 2003 92 827; Egeblad and Werb Nat. Rev. Cancer 2002 2 161).

The broad range of substrate specificities and expression patterns of MMPs results in their involvement in many different processes, both normal and pathological. The aberrant expression of MMPs has been noted in asthma, cancer, angiogenesis, rheumatoid arthritis, osteoarthritis, osteoporosis, intestinal inflammatory diseases, periodontal disease, atherosclerosis, emphysema, multiple sclerosis, pre-eclampsia, and chronic wounds, among other diseases and disorders (Bergers and Coussens Curr. Opin. Genet. Dev. 2000 10 120; Visse and Nagase Circ. Res. 2003 92 827; Nelson. et al. J. Clin. Oncol. 2000 18 1135).

The general structure of an MMP protein consists of a pre domain to direct secretion from the cell, a pro domain, a catalytic domain, and a C-terminal hemopexin domain. In order to function MMPs need to bind both Ca²⁺ and Zn²⁺ ions—only Zn²⁺ is bound in the active site of the enzyme, Ca²⁺ is only required for maintaining the molecule's conformation. The inactive, or zymogen, form of the enzyme is activated by proteolytic removal of the pro domain (Woessner and Nagase Metalloproteinases and TIMPs. 2000 Oxford University Press).

Based on structural and functional considerations of the known matrix metalloproteinases, MMPs have been classified into families and subfamilies as described in N. M. Hooper (1994) FEBS Letters 354:1-6.

Examples of matrix metalloproteinases (MMPs) include collagenases (MMP1, MMP8, MMP13), gelatinases (MMP2, MMP9), stromelysins (MMP3, MMP10, MMP11), matrilysin (MMP7), metalloelastase (MMP12), enamelysin (MMP19), the MT-MMPs (MMP14, MMP15, MMP16, MMP17).

Preferably the matrix metalloproteinase is selected from the group consisting of MMP1 (EC 3.4.24.7), MMP2 (EC3.4.24.24), MMP3 (EC 3.4.24.17), MMP8 (EC 3.4.24.34), MMP9 (EC 3.4.24.35), MMP12 (EC3.4.24.65) and MMP13 (EC 3.4.24.-).

More preferably the matrix metalloproteinase is selected from the group consisting of MMP8 (EC 3.4.24.34), MMP9 (EC 3.4.24.35), MMP12 (EC3.4.24.65) and MMP13 (EC3.4.24.-).

In a highly preferred embodiment said matrix metalloproteinase is MMP9 (EC 3.4.24.35).

In another highly preferred embodiment said matrix metalloproteinase is MMP12 (EC3.4.24.65).

In an additional highly preferred embodiment said matrix metalloproteinase is MMP13 (EC3.4.24.-).

MMP1 (EC 3.4.24.7)

MMP1, also known as collagenase (EC 3.4.24.7), was identified by Brinckerhoff et al (1987 J. Clin. Invest. 79: 542-546). Collagenase is the only enzyme able to initiate breakdown of the interstitial collagens, types I, II, and III. It can also cleave collagens of types VII and X. Collagens are the most abundant proteins in the body and collagenase is a ubiquitous enzyme.

UniProt accession numbers P03956 and P08156, detail polypeptide sequences having MMP1 activity.

As an example, P03956 cleaves of the triple helix of collagen at about three-quarters of the length of the molecule from the N-terminus, at 775-Gly-.

MMP2 (EC3.4.24.24)

MMP2, also known as gelatinase 2, is secreted as a pro-enzyme. The cleavage of the pro-enzyme leads to the production of a soluble active form that is further trapped by receptors present at the surface of cancer cells (Brooks P. et al., 1996, Cell, 31; 85(5):683-93). MMP2 activation from the pro- to the mature form is a complex mechanism involving the membrane-type MMPs (MT-MMPs). MT1-MMP (MMP14) is the most potent activator of MMP2. The extracellular activity of MMPs is inhibited when they form complexes with specific inhibitors, such as TIMP1 and TIMP2.

MMP2 cleaves gelatin type I and collagen types IV, V, VII, X.

UniProt accession number P08253 details a polypeptide sequence having MMP2 activity.

As an example, P08253 cleaves the collagen-like sequence Pro-Gln-Gly-|-Ile-Ala-Gly-Gln. It also cleaves KiSS1 at a Gly-|-Leu bond.

MMP3 (EC 3.4.24.17)

MMP3, (also called fibroblast stromelysin or transin) is a proteoglycanase closely related to collagenase (MMP1). MMP3 is a secreted metalloprotease produced predominantly by connective tissue cells. MMP3 has a wide range of substrate specificities and is capable of degrading proteoglycan, fibronectin, laminin, and type IV collagen, but not interstitial type I collagen.

UniProt accession numbers P08254 and Q6GRF8 detail polypeptide sequences having MMP3 activity.

As an example, P08254 can degrade fibronectin, laminin, gelatins of type I, III, IV, and V; collagens III, IV, X, and IX, and cartilage proteoglycans. It can also activate procollagenase. P08254 preferentially cleaves at hydrophobic residues.

MMP-3 activity has been demonstrated in fibroblasts isolated from inflamed gingiva [Uitto V. J. et al, 1981, J. Periodontal Res., 16:417-424], and enzyme levels have been correlated to the severity of gum disease [Overall C. M. et al, 1987, J. Periodontal Res., 22:81-88]. MMP-3 is also produced by basal keratinocytes in a variety of chronic ulcers [Saarialho-Kere U. K. et al., 1994, J. Clin. Invest., 94:79-88].

MMP-3 mRNA and polypeptides were detected in basal keratinocytes adjacent to but distal from the wound edge in what probably represents the sites of proliferating epidermis. MMP-3 may thus prevent the epidermis from healing.

Several investigators have demonstrated consistent elevation of MMP-3 in synovial fluids from rheumatoid and osteoarthritis patients as compared to controls [Walakovits L. A. et al, 1992, Arthritis Rheum., 35:35-42; Zafarullah M. et al, 1993, J. Rheumatol., 20:693-697].

MMP8 (EC 3.4.24.34)

MMP-8 (collagenase-2 or neutrophil collagenase) is preferentially expressed in neutrophils. Studies indicate MMP-8 is expressed also in other cells, such as osteoarthritic chondrocytes [Shlopov et al, 1997, Arthritis Rheum, 40:2065].

UniProt accession numbers P22894 and Q45F99 detail polypeptide sequences having MMP8 activity.

As an example, P22894 can degrade fibrillar type I, II, and III collagens. Furthermore, it cleaves of interstitial collagens in the triple helical domain.

MMPs produced by neutrophils can cause tissue remodelling, and hence blocking MMP-8 should have a positive effect in fibrotic diseases of for instance the lung, and in degradative diseases such as pulmonary emphysema.

MMP-8 has also been found to be up-regulated in osteoarthritis, indicating that blocking MMP-8 many also be beneficial in this disease.

MMP9 (EC 3.4.24.35)

MMP9 (Gelatinase B; 92 kD Type IV Collagenase; 92 kD Gelatinase) is a secreted protein which was first purified, then cloned and sequenced, in 1989 [S. M. Wilhelm et al (1989) J. Biol. Chem. 264 (29): 17213-17221; published erratum in J. Biol. Chem. (1990) 265 (36): 22570].

UniProt accession numbers P14780, Q8N725, Q9H4Z1 and Q3LR70 detail polypeptide sequences having MMP9 activity.

As an example, P14780 cleaves KiSS1 at a Gly-. P14780 cleaves gelatin types I and V and collagen types IV and V.

Vu and Werb (1998) review the protease MMP9 (In: Matrix Metalloproteinases. 1998. Edited by W. C. Parks & R. P. Mecham. pp 115-148. Academic Press. ISBN 0-12-545090-7). The following points are drawn from this review by T. H. Vu & Z. Werb (1998).

The expression of MMP9 is restricted normally to a few cell types, including trophoblasts, osteoclasts, neutrophils and macrophages. However, the expression of MMP9 can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins.

MMP9 has been implicated in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues.

Increased MMP9 expression has been observed in, such as COPD, asthmatics, arthritis, tumour metastasis, Alzheimer's, Multiple Sclerosis, and plaque rupture in atherosclerosis leading to acute coronary conditions such as Myocardial Infarction thereby implicating MMP9 in disease processes.

MMP12 (EC3.4.24.65)

MMP12, also known as macrophage elastase or metalloelastase, was initially cloned in the mouse by Shapiro et al (1992, Journal of Biological Chemistry 267: 4664) and in man by the same group in 1995 (Belaaouaij et al 1995, J biol Chem 270:14568-14575).

UniProt accession number P39900 details a polypeptide sequence having MMP12 activity.

MMP12 is preferentially expressed in activated macrophages, and has been shown to be secreted from alveolar macrophages from smokers (Shapiro et al, 1993, Journal of Biological Chemistry, 268: 23824) as well as in foam cells in atherosclerotic lesions (Matsumoto et al, 1998, Am J Pathol 153: 109).

A mouse model of COPD is based on challenge of mice with cigarette smoke for six months, two cigarettes a day six days a week. Wildtype mice developed pulmonary emphysema after this treatment. When MMP12 knock-out mice were tested in this model they developed no significant emphysema, indicating that MMP112 is a key enzyme in the COPD pathogenesis.

The role of MMPs such as MMP12 in COPD (emphysema and bronchitis) is discussed in Anderson and Shinagawa, 1999, Current Opinion in Anti-inflammatory and Immunomodulatory Investigational Drugs 1(1): 29-38.

Matetzky et al (2000) disclose that smoking increases macrophage infiltration and macrophage-derived MMP-12 expression in human carotid artery plaques Kangavari (Natetzky S, Fishbein M C et al., Circulation 102:(18), 36-39 Suppl. S, Oct. 31, 2000).

MMP13 (EC 3.4.24.-)

MMP13, or collagenase 3, was initially cloned from a cDNA library derived from a breast tumour (J. M. P. Freije et al. (1994) Journal of Biological Chemistry 269(24):16766-16773). UniProt accession numbers P45452 and Q6NWN6 detail polypeptide sequences having MMP13 activity.

Analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes (Johansson et al., 1997 Cell Growth Differ. 8(2):243-250) squamous cell carcinomas (Johansson et al., (1997) Am. J. Pathol. 151(2):499-508) and epidermal tumours (irola et al., (1997) J. Invest. Dermatol. 109(2):225-231). These results suggest that MMP13 is secreted by transformed epithelial cells and may be involved in the extracellular matrix degradation and cell-matrix interaction associated with metastasis especially as observed in invasive breast cancer lesions and in malignant epithelia growth in skin carcinogenesis.

Recent published data implies that MMP13 plays a role in the turnover of other connective tissues. For instance, consistent with the substrate specificity of MMP13 and preference for degrading type II collagen (Mitchell et al., 1996 J. Clin. Invest. 97(3):761-768; Knauper et al., 1996 The Biochemical Journal 271:1544-1550), MMP13 has been hypothesised to serve a role during primary ossification and skeletal remodelling (Stahle-Backdahl et al., 1997 Lab. Invest. 76(5:717-728; Johansson et al., 1997 Dev. Dyn. 208(3):387-397), in destructive joint diseases such as rheumatoid and osteo-arthritis (Wernicke et al., 1996 J. Rheumatol. 23:590-595; Mitchell et al., 1996 J. Clin. Invest. 97(3):761-768; Lindy et al., 1997 Arthritis Rheum 40(8):1391-1399); and during the aseptic loosening of hip replacements (Imai et al., 1998 J. Bone Joint Surg. Br. 80(4):701-710). MMP13 has also been implicated in chronic adult periodontitis as it has been localised to the epithelium of chronically inflamed mucosa human gingival tissue (V. J. Uitto et al., 1998 Am. J. Pathol 152(6):1489-1499) and in remodelling of the collagenous matrix in chronic wounds (Vaalamo et al., 1997 J. Invest. Dermatol. 109(1):96-101).

### Substrate and Reporter

The term “substrate” as used herein refers to a compound having the formula (1)-viz compounds of the general formula (1a), (1b), (1c) or (1d).

The term “reporter” as used herein refers to a compound having the formula H—X—R₁; wherein R₁ is a hydrocarbyl group; R₂ is a first peptide moiety; R₃ is a second peptide moiety; and X is selected from the group consisting of O, S and NH.

Preferably X is S.

In an alternative embodiment, preferably X is NH.

Preferably the hydrocarbyl group R₁ is selected from the group consisting of an aryl, heteroaryl, aryloxyaryl, biaryl, alkyl, cycloalkyl, heterocycloalkyl group and derivatives thereof.

More preferably R₁ is selected from the group consisting of an aryl, heteroaryl, aryloxyaryl, biaryl and derivatives thereof.

In a highly preferred embodiment R₁ is selected from panel 1 wherein X in panel 1 denotes the remainder of the substrate.

In a highly preferred embodiment R₁ is selected from panel 1 wherein X in panel 1 denotes the remainder of the reporter having the formula H—X—R₁.

Hydrocarbyl Group

The term “hydrocarbyl group” as used herein means a group comprising at least C and H and may optionally comprise one or more other suitable substituents. Examples of such substituents may include halo-, alkoxy-, hydroxy-, nitro-, a hydrocarbon group, an amide group, a carbamate ester group, an ester group such as an acyl group, a nitrile group, an N-acyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable heteroatoms will be apparent to those skilled in the art and include, for instance, sulphur, nitrogen, oxygen, silicon and phosphorus.

For example, the hydrocarbyl group may be selected from an aryl, aryloxyaryl, biaryl, alkyl, cycloalkyl, heterocylic group and derivatives thereof. For example, the hydrocarbyl group may be selected from a pyridyl, or a pyrimidyl.

Hydrocarbon Group

Here the term “hydrocarbon” means any one of an alkyl group, an alkenyl group, an alkynyl group, an acyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

Heterocyclic Group

Herein the term “heterocyclic group” means a cyclic ring comprising at least one carbon atom and at least one heteroatom in the ring. The heterocyclic group may be heterocycloalkyl group or an heteroaryl group. Suitable heteroatoms will be apparent to those skilled in the art and include, for instance, sulphur, nitrogen, oxygen, silicon and phosphorus.

The heterocyclic ring may optionally be substituted with one or more suitable substitutents. Examples of such substituents may include halo-, alkoxy-, a halogen substituted alkoxy-, hydroxy-, nitro-, a hydrocarbon group, an amide group, a carbamate ester group, an alkyl carbamate ester group, an ester group, a nitrile group, an N-acyl group, or a cyclic group such as an aryl group. In addition to the possibility of the substituents being a cyclic group, a combination of substituents may form a cyclic group.

Chemical Derivative

In one embodiment of the present invention, the compound may be a derivative.

The term “derivative” as used herein includes chemical modification of a compound. Illustrative of such chemical modifications would be replacement of hydrogen by halo group, alkoxy group, a halogen substituted alkoxy group, a hydroxyl group, a nitro group, a hydrocarbon group, an amide group, a carbamate ester group, an alkyl carbamate ester group, an ester group such as an acyl group, a nitrile group, an N-acyl group, or a cyclic group such as an aryl group.

Substituents

The compounds of the present invention may have substituents other than those of the ring systems shown herein. Furthermore the ring systems herein are given as general formulae and should be interpreted as such. The absence of any specifically shown substituents on a given ring member indicates that the ring member may be substituted with any moiety of which H is only one example. The ring system may contain one or more degrees of unsaturation, for example in some aspects one or more rings of the ring system is aromatic. The ring system may be carbocyclic or may contain one or more hetero atoms.

Peptidic Moiety

The first peptide moiety (R₂) and second peptide moiety (R₃) comprise peptidic moieties.

As used herein, a peptidic moiety is a group comprising one or more amino acids attached by a peptide bond. The amino acid(s) may be natural amino acid(s) or unnatural amino acid(s) or combinations thereof. Preferred examples of amino acids are α-amino acid units. The peptidic moiety may further comprise one or more β-, γ-, ω- or δ-amino acid units. The amino acid units may be capped at one end by an end group, a protecting group, a chromophoric group, or a fluorophoric group.

If the substrate carries a chromophoric or fluorophoric group in addition to the reporter group, then the substrate can be used to assay the cleavage at any bond and a specific bond simultaneously or separately in the same sample.

Preferably R₂ is a dipeptide, a tripeptide or tetrapeptide.

Preferably R₃ is a dipeptide, a tripeptide or tetrapeptide.

Preferably R₂ is a tripeptide S3-S2-S1 where S3 is selected from proline or glycine, S2 is selected from leucine, cyclohexylalanine, phenylalanine, tyrosine and S1 is selected from glycine, alanine, serine and histidine.

More preferably R₂ is proline-leucine-glycine.

Alternatively, more preferably R₂ is glycine-leucine-alanine.

Preferably R₃ is a dipeptide S2′-S3′ where S2′ is selected from leucine, cyclohexylalanine, phenylalanine, tyrosine and S3′ is selected from glycine, β-alanine or is omitted.

More preferably R₃ is leucine-β-alanine.

End Groups

In some embodiments, the substrate may comprise an end group to eliminate the charge at the N-terminus and/or C-terminus. Such an end group may also be a specifically removable protecting group. Suitable end groups and protecting groups will be readily apparent to one skilled in the art. Such end groups and protecting groups and the methods of adding and/or removing them from the substrate may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

Suitable N-terminal endgroups (G1 in formulas 1c and 1d) include acetyl, benzoyl, tert-butyloxycarbonyl, benzyloxycarbonyl and fluorenylmethyloxycarbonyl. Suitable C-terminal endgroups (G2 in formulas 1c and 1d) include methyl ester, ethyl ester, tert-butyl ester, methylamide, ethylamide and phenylamide.

Preparation of the Substrate

Substrate compounds of the present invention may be prepared by standard chemical techniques.

For example, synthetic blocks of formula II can be prepared—such as by techniques described by Paulitz et al, J. Org. Chem., 1997, 62(24), 8474-8—and then used to make compounds of the formula (1).

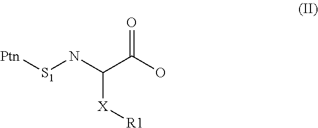

wherein:
   R₁ is a hydrocarbyl group or a polystyrene polymer
   S1 is selected from glycine, alanine, serine and histidine, optionally protected when appropriate
   Ptn is an end group, preferably a protecting group
   X is selected from the group consisting of O, S and NH;

Alternatively, their preparation can be adapted to the attachment of sulfhydryl-derivatized polymers, preferably polystyrene.

Preferably, synthetic blocks of formula II can be prepared by reacting N-protected amino acid amides Ptn-S1-NH₂ with glyoxylic acid and treatment of the intermediate with thiols R1-SH and an acid catalyst such as sulphuric acid:

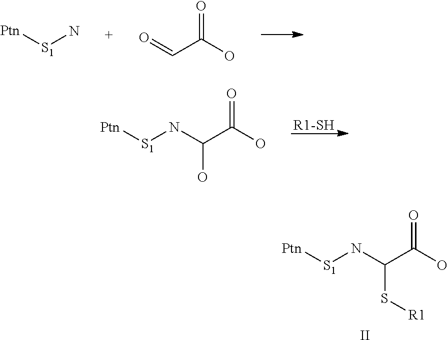

The building blocks of formula II can be soluble and/or polymeric.

Preferably, Ptn is fluorenylmethyloxycarbonyl (Fmoc).

The building blocks of formula II can be incorporated into full-length precursors of the substrate of formula Ie by standard methods of peptide synthesis. The general principles of peptide synthesis as well as amino acid side-chain protection strategies apply and are well known to those skilled in the art. General textbooks on the subject include “Principles of Peptide Synthesis” by M. Bodansky (Springer-Verlag 1984) and “Solid Phase Peptide Synthesis: A Practical Approach” by E. Atherton and R. C. Shepperd (IRL Press, Oxford University Press 1989).

a) Solid-phase peptide synthesisers on linked solid supports are used to prepare the substrate. The N-protecting group is preferably Fmoc; Pol is a polymeric support, preferably polystyrene beads, G₁, G₂, L₁, L₂, S₁, S₂, S₃, S₂′, S₃′ are as defined in formula 1d.

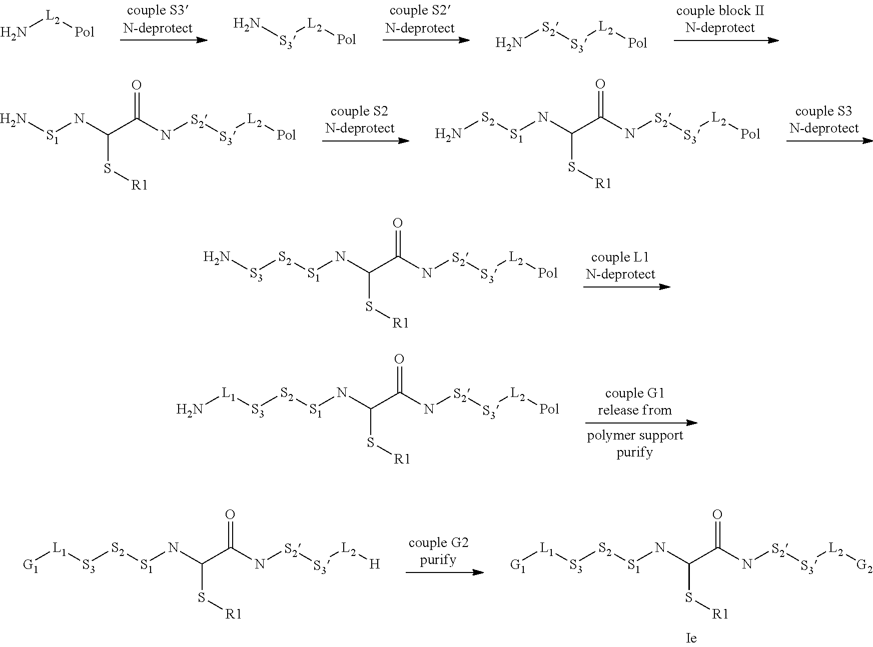

b) Solution synthesis of mono, di, tri and tetrapeptide blocks etc are used to produce the substrate. The N-protecting group Ptn is preferably Fmoc; G₁, G₂, L₁, L₂, S₁, S₂, S₃, S₂′, S₃′ are as defined in formula 1d.

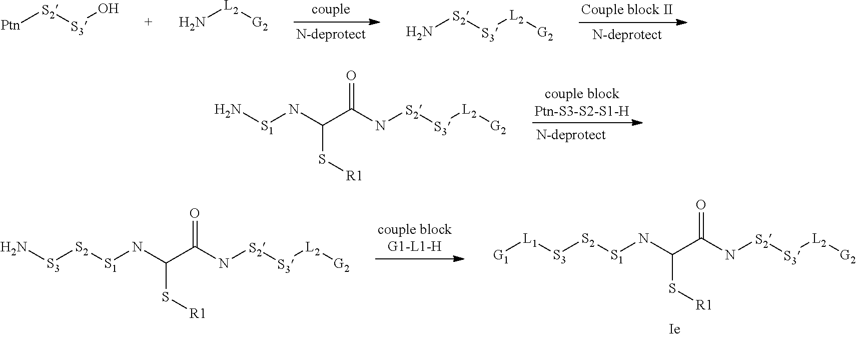

c) Alternatively, when R₁ is a solid support, such as polystyrene, the synthetic block of formula II can be grown in both directions. The N-protecting group Ptn is preferably Fmoc; G₁, G₂, L₁, L₂, S₁, S₂, S₃, S₂′, S₃′ are as defined in formula 1d.

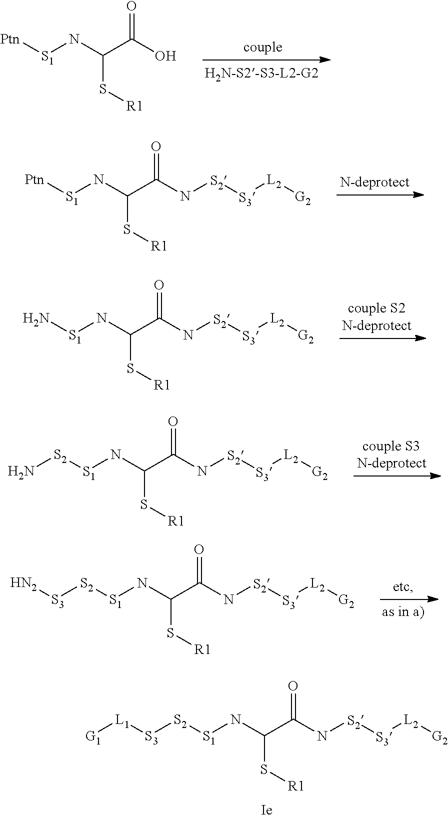

This results in the formation of peptides of formula 1c, where R₁ is preferably a polystyrene polymer. Alternatively, R₁ is the reporter substituent selected from the structures in Plate 1, in which case the peptide constitutes a substrate of formula 1d where X is S.

The N-protecting group Ptn is preferably Fmoc; G₁, G₂, L₁, L₂, S₁, S₂, S₃, S₂′, S₃′ are as defined in formula 1d.

When the peptide has the required sequence and appropriate end-group capping (if required), it is released from solid support by standard methods when method a) is used and eventually modified at the cleavage end, or products of methods b) and c) are (in step d) treated with a thiophilic reagent, preferably N-iodosuccinimide (NIS) and are reacted with excess alcohols, phenols, thiols or amines of formula H—X—R₁ to obtain the substrate of formula 1d. These are preferably separated to their diastereomers, since the peptides where the stereochemistry of the —XR₁, residue corresponds to that of the natural substrates are expected to be cleaved very efficiently compared to the other stereoisomer.

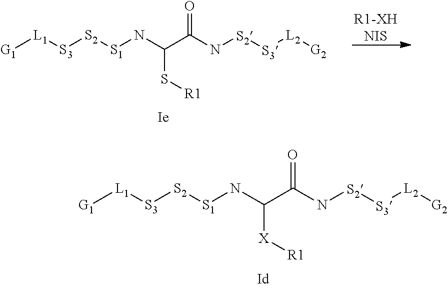

If further modifications are desired after the introduction of —XR₁, the required protecting group manipulations are preferably non-acidic conditions and coupling reactions.

Alternatively, the R₁—X group can be exchanged via the chloro or bromo derivative (1f):

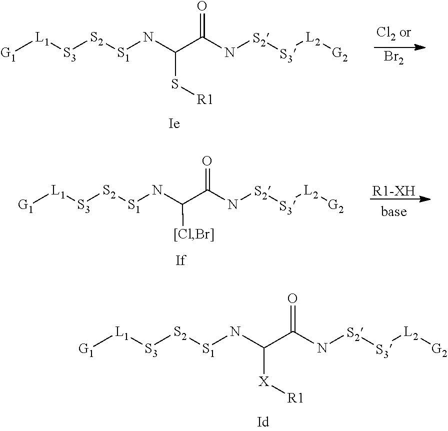

Optionally, if the substituent XR₁, of formula (1) is SR₁, of formula II and III, the thiol exchange reaction in step (d) is not necessary.

Cleavage of the Substrate to Produce the Reporter

The substrate of the present invention having the formula (1) is capable of being cleaved by a protease. In other words the substrate is capable of being enzymatically digested by a protease.

As a result of a substrate having formula (1) being cleaved by a protease a reporter having the formula H—X—R₁ is formed. This enzymatic reaction is represented as follows:

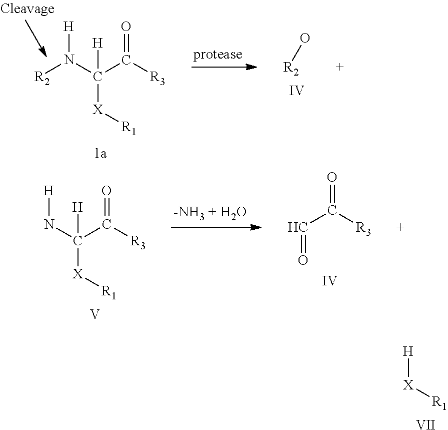

The cleavage arrow represents the protease action.

When the substrate having the formula (1) is cleaved at the bond of interest, the resulting intermediate compound (V) is an unstable intermediate when X is a heteroatom (O, S, or NH). The amino group in the intermediate compound V is an electron-donating group and destabilises the bond to the side chain —XR₁, which is rapidly hydrolysed. This results in the formation of compound VII (i.e. the reporter).

If a protease cleaves the substrate elsewhere then compound VII (i.e. the reporter) is not produced.

Thus X and R₁ of the substrate will be the same as X and R₁ of the reporter.

The term “release” as used herein refers to the production of the reporter as described in the above-mentioned enzymatic reaction following enzymatic digestion of a substrate as described herein by a protease.

The term “bond of interest” as used herein refers to a bond in the substrate of formula (I) which is cleaved by a protease and which results in the formation of the reporter H—X—R₁. The bond of interest is between a carbonyl group and the NH—CH(X—R₁) group.

Detection of the Reporter

The term “activity of a protease” as used herein refers to the enzymatic activity of the protease.

The activity of a protease in a sample can be determined by admixing the sample with a substrate having the formula (1) and detecting the presence of the reporter having the formula H—X—R₁.

Preferably the admixture is incubated for between 5 and 120 minutes, more preferably said reaction is incubated for about 30 minutes, in a highly preferred embodiment said reaction is incubated for about 15 minutes once the substrate having formula (1) has been added to the sample.

Preferably the admixture is incubated at a temperature between 15 to 40° C. More preferably the admixture is incubated at about 37° C.

The presence in a sample of a reporter having the formula H—X—R₁ may be detected by HPLC, LC-UV, LC-MS or fluorescence measurements (if R₁ is a fluorophore and there is a quenching group elsewhere in the substrate or vice versa).

Hence the term “means for detecting a reporter” as used herein refers to suitable means, such as HPLC and LC-MS, for detecting in a sample the presence of a reporter having the formula H—X—R₁.

HPLC and LC-MS are techniques which are well known in the art (see, for example, Snyder and Kirkland—Introduction to Modern Liquid Chromatography, Second edition, John Wiley & Sons, Inc. 1979 (HPLC); and Jurgen H Gross—Mass Spectrometry, A textbook, Springer Verlag 2004 (LC-MS)).

In order to determine the amount of activity of a protease in a sample, the amount of a reporter having the formula H—X—R₁ produced in a given time period in a sample admixed with the substrate having formula (1) can be compared to the amount of reporter having the formula H—X—R₁, produced in a given time period in a control sample admixed with the substrate having formula (1) and treated with known amounts of a protease. Preferably the admixture is incubated between 5 and 120 minutes, more preferably said reaction is incubated for about 30 minutes, in a highly preferred embodiment said reaction is incubated for about 15 minutes once the substrate having formula (1) has been added to the sample.

A sample which has been admixed with a substrate having the formula (1) will not produce a reporter having the formula H—X—R₁ if there is no protease in the sample capable of acting on the bond of interest of the substrate having the formula (1). Hence there will be no reporter present in such a sample.

Preferably the substrate having formula (1) is methyl 1-acetyl-L-prolyl-L-leucylglycyl-α-R-(4-nitrophenylamino)-glycyl-L-leucyl-β-alaninate.

Here the reporter having formula H—X—R₁ is 4-nitroaniline.

Alternatively, preferably the substrate having formula (1) is methyl 1-acetyl-L-prolyl-L-leucylglycyl-α-R—[4-(5-p-tolyl-[1,3,4]oxadiazol-2-yl)-phenylamino]-glycyl-L-leucylglycinate.

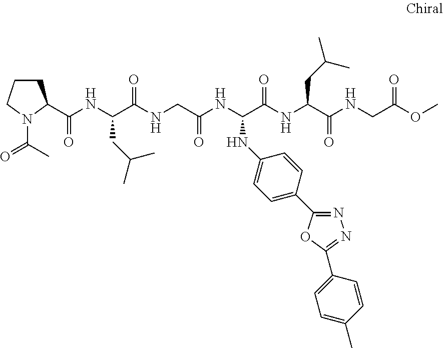

Here the reporter having formula H—X—R₁ is 4-(5-p-Tolyl-[1,3,4]oxadiazol-2-yl)-phenylamine.

Alternatively, preferably the substrate having formula (1) is methyl 1-acetyl-L-prolyl-L-leucylglycyl-α-R-(4-nitrophenylamino)-glycyl-N-phenyl-L-phenylalaninamide.

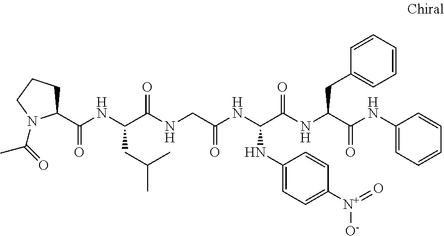

Here the reporter having formula H—X—R₁ is 4-nitroaniline.

Alternatively, preferably the substrate having formula (1) is methyl 1-acetyl-L-prolyl-L-leucylglycyl-α-S-(biphenyl-4-ylmethoxy)-glycyl-L-leucylglycinate.

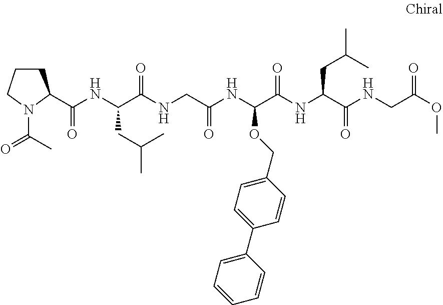

Here the reporter having formula H—X—R₁ is biphenyl-4-yl-methanol.

Alternatively, preferably the substrate having formula (1) is methyl 1-acetyl-L-prolyl-L-leucylglycyl-α-R-(4-nitrophenylamino)-glycyl-L-leucylglycinate.

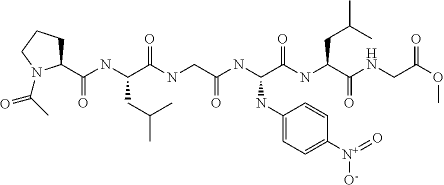

Here the reporter having formula H—X—R₁ is 4-nitroaniline.

### Protease-Modulators

The term “protease-modulator” as used herein refers to any compound or composition, such as a pharmaceutical composition, which is capable of:
(i) increasing the activity of a protease or increasing the release of a protease(s)—i.e. it is a protease-activator; or
(ii) decreasing the activity of a protease or decreasing the release of a protease(s)—i.e. it is a protease-inhibitor.

The protease-modulator may be an agonist or an antagonist.

Preferably the protease-modulator is a protease-activator.

An example of a protease-activator is zymosan. Without wishing to bound by theory, the addition of zymosan (yeast cell wall beta-glucan) to whole blood results in the activation and degranulation of intracellular granulas containing proteases such as MMP-8 and MMP-9.

Without wishing to be bound by theory, the activity of a protease may be increased by, for example, the binding of the protease-activator to the protease which enables the protease to bind more efficiently to its substrate and thus increase the amount of peptide cleavage when compared to a control sample. Alternatively, the protease-activator may increase the activity of a protease by, for example, cleaving the pro-protease into the protease. Alternatively the protease-activator may increase the release of a protease(s).

In an alternative embodiment, preferably the protease-modulator is a protease-inhibitor.

Without wishing to be bound by theory, in another embodiment the activity of a protease may be decreased by, for example, the binding of the protease-inhibitor to the protease which enables the protease to bind less efficiently to its substrate and thus decrease the amount of peptide cleavage when compared to a control sample. Alternatively, the protease-inhibitor may decrease the activity of a protease by inhibiting the cleavage of the pro-protease into the protease. Alternatively the protease-activator may decrease the release of a protease(s).

Preferably the protease-inhibitor is selected from the group consisting of MMP1 protease-inhibitors, MMP2 inhibitors, MMP3 protease-inhibitors, MMP8 protease-inhibitors, MMP9 protease-inhibitors, MMP12 protease-inhibitors and MMP13 protease-inhibitors. More preferably the protease-inhibitor is selected from the group consisting of MMP8 protease-inhibitors, MMP9 protease-inhibitors and MMP12 protease-inhibitors. In a highly preferred embodiment the protease inhibitor is an MMP9 protease inhibitor. In another highly preferred embodiment the protease inhibitor is an MMP13 protease inhibitor. In yet another highly preferred embodiment the protease inhibitor is an MMP12 protease inhibitor.

A number of metalloproteinase inhibitors are known (see for example the review of MMP inhibitors by Beckett R. P. and Whittaker M., 1998, Exp. Opin. Ther. Patents, 8(3):259-282). Different classes of compounds may have different degrees of potency and selectivity for inhibiting various metalloproteinases.

The term “candidate protease-modulator” as used herein refers to any composition, such as a pharmaceutical composition, which is being assessed for its ability to either increase the activity of a protease or decrease the activity of a protease.

Preferably the candidate protease-modulator is a candidate protease-inhibitor.

Preferably the candidate protease-inhibitor is selected from the group consisting of MMP1 candidate protease-inhibitors, MMP2 candidate protease-inhibitors, MMP3 candidate protease-inhibitors, MMP8 candidate protease-inhibitors, MMP9 candidate protease-inhibitors, MMP12 candidate protease-inhibitors and MMP13 candidate protease-inhibitors. More preferably the candidate protease-inhibitor is selected from the group consisting of MMP8 candidate protease-inhibitors, MMP9 candidate protease-inhibitors, MMP12 candidate protease-inhibitors and MMP13 candidate protease-inhibitors. In a highly preferred embodiment the candidate protease-inhibitor is an MM9 inhibitor. In another highly preferred embodiment the candidate protease inhibitor is an MMP13 protease inhibitor. In yet another highly preferred embodiment the candidate protease inhibitor is an MMP12 protease inhibitor.

In an alternative embodiment, preferably the candidate protease-modulator is a candidate protease-activator. More preferably the candidate protease-activator is selected from the group consisting of MMP8 candidate protease-activators, MMP9 candidate protease-activators and MMP12 candidate protease-activators.

Metalloproteinases are considered to play a critical role in normal development and physiological tissue remodeling and repair. In addition, they are considered to play an important role in the regulation of the kinetics and function of inflammatory cells. Metalloproteinases have been associated with many diseases or conditions (see, for example, Doherty et al, Expert Opin. Ther. Patents 2002; 12(5): 594-604). The inhibition of the activity of one or more metalloproteinases may well be of benefit in certain diseases or conditions.

Metalloproteinase inhibitors may be used in the treatment of various inflammatory diseases and diseases associated with uncontrolled degradation of the extracellular matrix and remodelling. Such diseases and disorders include: rheumatoid arthritis, osteoarthritis, gout, systemic lupus erythematosus (SLE), inflammation of the gastro-intestinal tract (especially inflammatory bowel disease, ulcerative colitis and gastritis), inflammation of the skin (especially psoriasis, eczema, dermatitis). Furthermore metalloproteinase inhibitors may be used in the treatment of: tumour growth and metastasis; bone resorptive diseases (such as osteoporosis and Paget's disease); diseases associated with aberrant angiogenesis; enhanced collagen remodelling associated with diabetes; periodontal disease (such as gingivitis); corneal ulceration; ulceration of the skin; post-operative conditions (such as colonic anastomosis); dermal wound healing; demyelinating diseases of the central and peripheral nervous systems (such as multiple sclerosis); Alzheimer's disease; extracellular matrix remodelling observed in cardiovascular diseases such as restenosis, atherosclerosis and aortic aneurisms; liver fibrosis; airway diseases such as asthma, rhinitis, chronic bronchitis, chronic obstructive bronchioliti, airway fibrosis and chronic obstructive pulmonary disease (COPD).

Chronic obstructive pulmonary disease (COPD) is a term for a group of respiratory tract diseases that are characterised by airflow obstruction or limitation. Conditions encompassed by the term “COPD” include chronic bronchitis, emphysema and bronchiectasis. COPD can cause tachycardia (rapid heart rate), seizures, comas, respiratory arrest and death. COPD is also characterised by exacerbations which typically present with a rapid progression of the chronic symptoms. Classically, an exacerbation is notable by increased shortness of breath, wheezing, and sputum production. COPD is usually caused by smoking.

### Samples

Preferably the sample is an ex vivo sample.

Preferably the sample is a biofluid.

Preferably the sample is a mammalian biofluid. More preferably the sample is selected from the group consisting of: urine, whole blood, blood plasma, blood serum, synovial fluid, saliva, sputum, bronchoalveolar fluids, cerebrospinal fluid, nasal lavage, lung lining fluid, tear fluid and skin blister fluid.

In an alternative embodiment, the sample is selected from the group consisting of: a tissue slice, a biopsy sample, a cell culture, and a homogenised tissue. More preferably the sample is a cell culture or a homogenised tissue.

In one embodiment, preferably the sample is obtained from subject treated with a protease-modulator.

In an alternative embodiment, preferably the sample is treated with a protease-modulator after the sample has been obtained from a subject.

In another embodiment, preferably the sample is obtained from subject treated with a candidate protease-modulator.

In an alternative embodiment, preferably the sample is treated with a candidate protease-modulator after the sample has been obtained from a subject.

As used herein the term “contacting a protease with a protease-modulator” refers to a sample which is treated with the protease-modulator. The sample may be obtained from a subject who has been treated with a protease-modulator—for example, a patient may have been on a course of a pharmaceutical composition which is a protease-inhibitor. Alternatively, the sample may be an ex vivo sample (such as a biofluid, a biopsy or a cell culture) which is admixed with the protease-modulator in vitro—for example, the sample may be obtained from a patient who has not been receiving a pharmaceutical composition which is a protease-modulator.

As used herein the term “contacting a protease with a candidate protease-modulator” refers to a sample which is treated with the candidate protease-modulator. The sample may be an ex vivo sample (such as a bodily fluid, a biopsy or a cell culture) which is admixed with the candidate protease-modulator in vitro—for example, the sample may be obtained from a patient who has not been receiving treatment with a pharmaceutical composition. Alternatively the sample may be obtained from a subject who has been treated with a candidate protease-modulator—for example, a patient may have been on a course of a pharmaceutical composition which is a candidate protease-inhibitor.

As used herein the term “determining the efficacy of a protease-modulator” refers to the comparison of sample(s) which have been contacted with a protease-modulator against sample(s) which have not been contacted with a protease-modulator and/or against sample(s) which have been contacted with a different protease-modulator.

A protease-inhibitor will result in a lower protease activity in a given sample in a given time period when compared to an untreated sample. In the present invention the activity of a protease in a sample is monitored by admixing the sample with a substrate having the formula (1) and detecting the production of the reporter H—X—R₁ in a given time period (for efficacy studies the reaction should not be allowed to proceed to completion). Hence a sample treated with a protease-inhibitor will result in the presence of less of the reporter in a given time period when compared to an untreated sample. Furthermore, a sample treated with a protease-inhibitor (the first protease-inhibitor) which results in the presence of less of the reporter in a given time period when compared with a sample treated with a different protease-inhibitor (the second protease-inhibitor) demonstrates that the first protease-inhibitor is a more effective protease-inhibitor than the second protease-inhibitor and vice versa.

A protease-activator will result in more protease activity in a given sample in a given time period when compared to an untreated sample. In the present invention the activity of a protease in a sample is monitored by admixing the sample with a substrate having the formula (1) and detecting the production of the reporter H—X—R₁, in a given period of time (for efficacy studies the reaction should not be allowed to proceed to completion). Hence a sample treated with a protease-activator will result in the presence of more of the reporter in a given time period than an untreated sample. Furthermore, a sample treated with a protease-activator (the first protease-activator) which results in the presence of more of the reporter in a given time period when compared with a sample treated with a different protease-activator (the second protease-activator) demonstrates that the first protease-activator is a more effective protease-activator than the second protease-activator and vice versa.

As used herein the term “determining the efficacy of a candidate protease-modulator” refers to the comparison of sample(s) which have been contacted with a candidate protease-modulator against sample(s) which have not been contacted with a candidate protease-modulator and/or against sample(s) which have been contacted with a known protease-modulator.

In the present invention the activity of a protease in a sample is monitored by admixing the sample with a substrate having the formula (1) and detecting the production of the reporter H—X—R₁, in a given time period (for efficacy studies the reaction should not be allowed to proceed to completion). Hence a sample treated with a desirable candidate protease-inhibitor will result in the presence of less of the reporter in a given time period when compared to an untreated sample. Furthermore, a sample treated with a candidate protease-inhibitor which results in the presence of less of the reporter in a given time period when compared with a sample treated with a known protease-inhibitor demonstrates that said candidate is a more effective protease-inhibitor and vice versa. However a sample treated with a desirable candidate protease-activator will result in the presence of more of the reporter in a given time period than an untreated sample. Furthermore, a sample treated with a candidate protease-activator which results in the presence of more of the reporter in a given time period when compared with a sample treated with a known protease-activator demonstrates that said candidate is a more effective protease-activator and vice versa.

As used herein the term “diagnosing a disease or disorder” as used herein refers to determining the level of activity of a protease in a sample from a subject and comparing the level of protease activity to the levels of protease activity in samples from subject(s) known to have the disease or disorder and in samples from subject(s) which do not have the disease or disorder. For example, the levels of MMP9 activity in a sample derived from a subject with COPD, a subject who smokes and a subject who does not smoke can be measured and compared. A subject who smokes and is in the early stages of COPD can thus be detected.

The methods as described herein can also be used for determining disease progression over a period of time in a subject.

Furthermore the methods as described herein can be used to determine the response of a subject to treatment with protease-modulators.

In another aspect, the methods as described herein can be used to determine the amount (i.e. dosage) of a protease-modulator which should be administered to a subject in order to achieve an advantageous effect such as stopping the progress of a disease or disorder or reversing the progression of a disease or disorder.

Preferably the disease or disorder is selected from the group consisting of: rheumatoid arthritis, osteoarthritis, gout, systemic lupus erythematosus (SLE), inflammation of the gastrointestinal tract (especially inflammatory bowel disease, ulcerative colitis and gastritis), inflammation of the skin (especially psoriasis, eczema, dermatitis), tumour growth, tumour metastasis; bone resorptive diseases (such as osteoporosis and Paget's disease); diseases associated with aberrant angiogenesis; enhanced collagen remodelling associated with diabetes; periodontal disease (such as gingivitis); corneal ulceration; ulceration of the skin; post-operative conditions (such as colonic anastomosis); dermal wound healing; demyelinating diseases of the central and peripheral nervous systems (such as multiple sclerosis); Alzheimer's disease; extracellular matrix remodelling observed in cardiovascular diseases such as restenosis, atherosclerosis and aortic aneurisms; liver fibrosis; airway diseases such as asthma, rhinitis, chronic bronchitis, chronic obstructive bronchioliti, airway fibrosis and chronic obstructive pulmonary disease (COPD).

Preferably the disease or disorder is selected from a group consisting of: rheumatoid arthritis, osteoarthritis; multiple sclerosis; airway diseases such as asthma, rhinitis, chronic bronchitis, chronic obstructive bronchioliti, airway fibrosis and chronic obstructive pulmonary disease (COPD).

More preferably the disease or disorder is osteoarthritis, asthma or chronic obstructive pulmonary disease (COPD).

In one highly preferred embodiment the disease or disorder is osteoarthritis.

In another highly preferred embodiment the disease or disorder is chronic obstructive pulmonary disease (COPD).

### EXAMPLES

The present invention is further described by way of examples and with reference to the following structures and Figures:

Structure of 4-nitroaniline

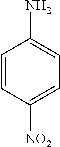

Structure of methyl 1-acetyl-L-prolyl-L-leucylglycyl-α(4-nitrophenylamino)-L-glycyl-L-leucyl-β-alaninate, highlighting the bond at which the substrate is cleaved in order to produce the reporter.

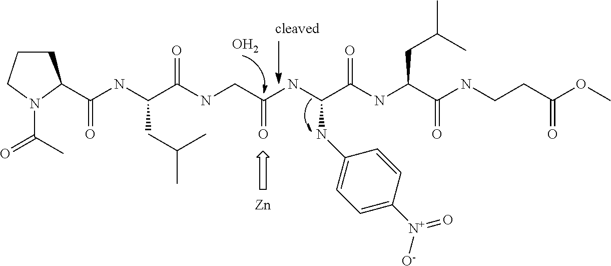

Structure of [¹³C]₆-4-nitroaniline

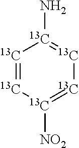

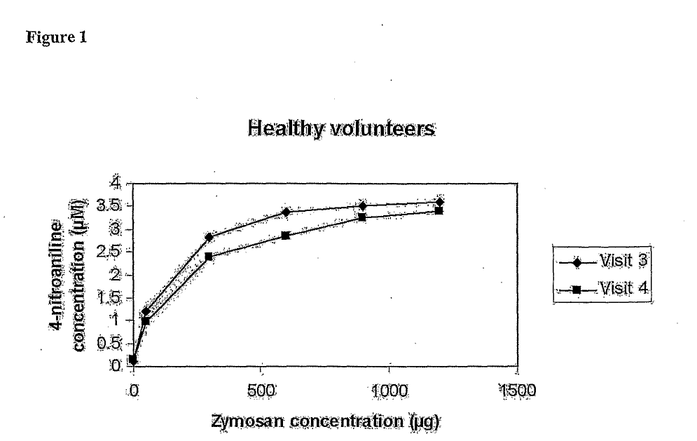

FIG. 1. Mean concentration profiles of 4-nitroaniline from healthy volunteers obtained after stimulation of whole blood with varying amounts of zymosan.

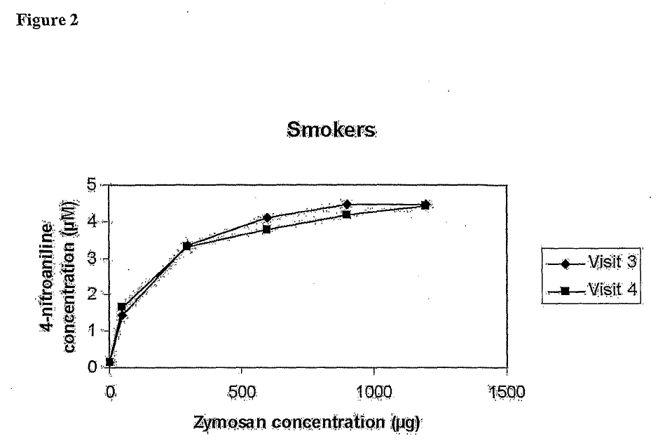

FIG. 2. Mean concentration profiles of 4-nitroaniline from smokers obtained after stimulation of whole blood with varying amounts of zymosan.

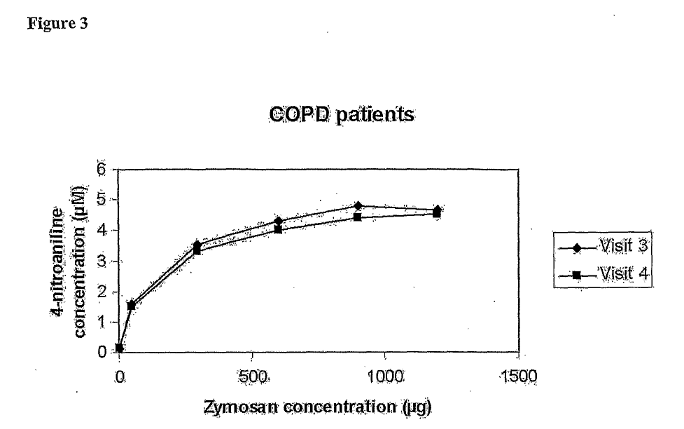

FIG. 3. Mean concentration profiles of 4-nitroaniline from COPD patients obtained after stimulation of whole blood with varying amounts of zymosan.

Abbreviations used in the examples include:

### DCM—Dichloromethane;

THF—tetrahydrofuran;
MeCN—acetonitrile;
MeOH—methanol;

### DMF—N,N-dimethylformamide;

EtOAc—ethylacetate;

### IPA—2-Propanol;

Et₂O—diethylether;
DMSO-D6—deuterated dimethyl sulfoxide;
CD₃CN—deuterated acetonitrile;
CD₃OD—deuterated methanol;

### TFA—Trifluoroaceticacid;

TfOH—Trifluoromethanesulfonic acid;
AcOH—acetic acid;

### DIEA—N-ethyldiisopropylamine;

HATU—O-(7-Azabenzotriazol-1-yl)-N,N,N′,N′-tetramethyluronium hexafluorophosphate;
LC/MS—liquid chromatography/mass spectrometry;
TLC—thin layer chromatography.

### Example 1

### Preparation of Substrates

### Material and Methods

In the Example, ¹H-NMR and ¹³C-NMR spectra were recorded on either a Varian ^{Unity}Inova 400 MHz or Varian Mercury-VX 300 MHz instrument. The central solvent peak of dimethylsulfoxide-d₆ (δ_{H} 2.50 ppm, δ_{C} 39.5 ppm), acetonitrile-d₃ (δ_{H} 1.95 ppm, δ_{C} 118.2, 1.3 ppm), methanol-d₄ (δ_{H} 3.31 ppm, δ_{C} 49.0 ppm) or pyridine-d₅ (δ_{H} 8.71, 7.55, 7.19 ppm, δ_{C} 149.9, 135.5, 123.5 ppm) were used as internal references.

Low resolution mass spectra were obtained on a Agilent 1100 LC-MS system equipped with an APCI ionization chamber or an ES ionization chamber.

Thin layer chromatography was made using TLC plates obtained from Merck with Silica gel 60 F₂₅₄ absorbed on glass plates. Seebach solution was used to visualise the TLC spots and was prepared from 25 g Phosphomolybdic acid, 10 g Ce(SO₄)₂.H₂O, 60 mL conc. H₂SO₄ and 940 mL H₂O.

Silica gel 60 with particle size 0.040-0.063 mm was obtained from Merck.

HPLC was carried out using Gilson analytical or semipreparative system. The conditions used in the example are as follows.
HPLC system A: Kromasil KR-100-7-C18, 250×20 mm column, isocratic solvent system of 62% MeOH/H₂O, solvent flow of 6 mL/min. and UV=220 nm for detection.
HPLC system B: Kromasil 100-C18-5 μm 150×4.6 mm column. Solvent A: H₂O/MeOH (=80/20) B: MeOH. 20 minutes gradient of 20% B to 90% B was used, flow 0.6 mL/min and UV=220 nm for detection.
HPLC system C: Kromasil 100-C18-5 μm 150×4.6 mm column. Solvent A: H₂O+0.1% TFA B: MeCN+0.1% TFA. 15 minutes gradient of 10% B to 90% B was used, flow 1 mL/min and UV=220 nm for detection.
HPLC system D: Kromasil 100-5C-18, 250×20 mm, flow: 6.0 ml/min., eluent: 75% MeOH, 25% H₂O, UV=220 nm.
HPLC system E: Kromasil KR-100-5-C18, 250×20 mm column, isocratic solvent system of 82% MeOH/H₂O, solvent flow of 6 mL/min. and UV=220 nm for detection.
HPLC system F: Kromasil 100-C18-5 μm 150×4.6 mm column, isocratic solvent system of 80% MeOH/H₂O, solvent flow of 1 mL/min. and UV=220 nm for detection.
HPLC system G: Kromasil KR-100-5-C18, 250×20 mm column, isocratic solvent system of 85% MeOH/H₂O, solvent flow of 6 mL/min. and UV=220 nm for detection.
HPLC system H: Kromasil 100-C18-5 μm 150×4.6 mm column, isocratic solvent system of 85% MeOH/H₂O, solvent flow of 1 mL/min. and UV=220 nm for detection.
HPLC system I: Kromasil 100-C18-5 μm 150×4.6 mm column, 30 min gradient, 5% MeCN/H₂O+0.1% TFA to 100% MeCN+0.1% TFA, solvent flow of 1 mL/min. and UV=220 nm for detection.
HPLC system J: Kromasil 100-C18-10 μm 250×50 mm column, isocratic 52% MeCN/H₂O, solvent flow of 50 mL/min. and UV=254 nm for detection.
HPLC system K: Kromasil 100-C18-5 μm 150×4.6 mm column, 20 min gradient, 5% MeCN/H₂O to 60% MeCN then isocratic 60% MeCN, solvent flow of 1 mL/min. and UV=254 nm for detection.

All solvents and commercial reagents were laboratory grade and used as received. Non-commercially available reagents was synthesised using procedures known in the art.

### Example 1.1

### Preparation of the Substrate: Methyl 1-acetyl-L-prolyl-L-leucylglycyl-α-R-(4-nitrophenylamino)-glycyl-L-leucyl-β-alaninate

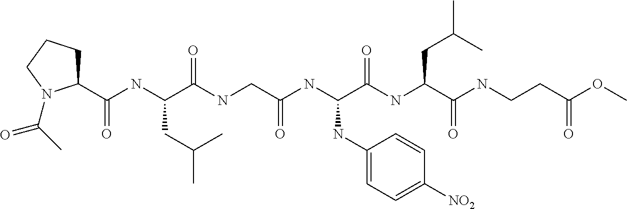

### a) 9H-fluoren-9-ylmethyl(2-amino-2-oxoethyl carbamate. (=Fmoc-Gly-NH₂)

Glycinamide hydrochloride (11.05 g; 100 mmol) and NaHCO₃ (16.8 g; 200 mmol) was dissolved in water (150 mL), when no more CO₂ (g) was formed a clear solution was obtained. Acetone (100 mL) was added and the solution was cooled on an ice/water bath. Fmoc-N-Hydroxysuccinimide (32 g; 95 mmol) was dissolved in acetone (400 mL) and added slowly to the reaction mixture, a slurry was formed. After the addition was completed the cold bath was removed and the slurry was stirred at room temperature for 3 days. Solvent was removed by evaporation and the residual colourless solid was suspended in water (1 L). The slurry was stirred for 1 hour, the solid product was collected by filtration and washed with water (2×500 mL). Dried under reduced pressure at +30° C. to constant weight.

28.1 g (100% yield) of the subtitled was obtained as a colourless solid.

HPLC system C: Rₜ=10.09 min, purity 97%.

APCI-MS m/z: 297.2 [MH⁺]

¹H-NMR (CD₃OD): δ 7.80 (2H, d), 7.67 (2H, d), 7.39 (2H, t), 7.31 (2H, t), 4.39 (2H, d), 4.23 (1H, t), 3.76 (2H, s) ppm.

### b) N-[(9H-fluoren-9-ylmethoxy)carbonyl]glycyl-α-R,S-(ethylthio)-glycine

Fmoc-Gly-NH₂ (14 g; 47 mmol) and Glyoxylic acid monohydrate (8.7 g; 94 mmol) was suspended in Acetone (400 mL) and H₂O (5 mL). The slurry was heated to reflux to form a slightly opaque solution. After 48 hours another portion of Glyoxylic acid monohydrate (8.7 g; 94 mmol) was added and the reflux was continued for another 24 hours. Evaporation of acetone gave an oil that was treated with H₂O (200 mL) and the resulting slurry was extracted with EtOAc (3×200 mL). The organic phase was then extracted with 5% NaHCO₃ (aq) (400 mL+200 mL+100 mL). The organic phase was discarded. The basic waterphase containing the desired intermediate N-[(9H-fluoren-9-ylmethoxy)carbonyl]glycyl-α-R,S-(hydroxy)-glycine was carefully acidified to pH 1-1.5 using conc. HCl, a slurry was formed. The acidic water phase was extracted with EtOAc (4×200 mL), the organic phase was washed with brine and dried over Na₂SO₄, filtered and evaporated to give a foamy oil. This oil was treated with DCM (200 mL) and the resulting solid material was collected by filtration. The solid material was redissolved in MeOH and THF and evaporated to give a crude product of N-[(9H-fluoren-9-ylmethoxy)carbonyl]glycyl-α-R,S-(hydroxy)-glycine. This crude product was not further purified, it was used directly as obtained to the next step. Obtained 14.4 g of the crude intermediate as a colourless solid. Purity was determined using HPLC system C: Rₜ=9.56 min, purity 68%.

Crude N-[(9H-fluoren-9-ylmethoxy)carbonyl]glycyl-α-R,S-(hydroxy)-glycine (14.4 g) was dissolved in AcOH (250 mL) and conc. H₂SO₄ (2.5 mL). Ethanethiol (15 mL; 0.2 mol) was added to the stirred solution at room temperature. After 21 hours the reaction mixture was slowly poured into 1 L of crushed ice and a solid was formed, when the ice had melted a slurry was obtained. The solid product was collected by filtration and washed with water (250 mL). The solid material was then washed with Et₂O followed by Heptane and finally Et₂O again before it was dried to constant weight under reduced pressure.

Obtained 10.2 g (52%) of the subtitle compound as a colourless solid.

HPLC system C: Rₜ=11.46 min, purity 92.5%.

APCI-MS m/z: 415.2 [MH⁺]

¹H-NMR (DMSO-D6): δ 13.23 (1H, brs), 8.63 (1H, d), 7.89 (2H, d), 7.71 (2H, d), 7.55 (1H, t), 7.42 (2H, t), 7.33 (2H, t), 5.36 (1H, d), 4.28 (2H, d), 4.23 (1H, t), 3.70 (2H, d), 2.62 (2H, m), 1.78 (3H, t) ppm.

¹³C-NMR (DMSO-D6): δ 169.60, 168.59, 156.26, 143.65, 140.53, 127.47, 126.92, 125.08, 119.96, 65.66, 52.76, 46.58, 43.07, 23.93, 14.49 ppm.

### c) Methyl 1-acetyl-L-prolyl-L-leucylglycyl-α-R,S-(ethylthio)-glycyl-L-leucyl-β-alaninate

The subtitle compound was partly synthesised on solid phase using Pioneer™ Peptide Synthesis System, with Tenta Gel S PHB Leu Fmoc as the starting point. Standard peptide coupling protocol developed for use with Fmoc-amino acids was followed.

N-[(9H-fluoren-9-ylmethoxy)carbonyl]glycyl-α-R,S-(ethylthio)-glycine was activated with 1,3-Diisopropylcarbodiimide. Commercially available Fmoc-L-Leu-OPfp and Fmoc-L-Pro-OPfp were used. N-acylation was made using a DMF solution containing 25% acetic acid anhydride and 5% Pyridine. 1-acetyl-L-prolyl-L-leucylglycyl-α-R,S-(ethylthio)-glycyl-L-leucine was cleaved of the resin using 95% TFA/H₂O and lyophilised. Final coupling with β-alanine methyl ester was done with HATU and DIEA in DMF. Purification of final peptide was made on a Gilson preparative HPLC system with a Kromasil KR-100-7-C18, 250×50.8 mm column. Final peptide was lyophilised and analysed using an Agilent 1100 LC-MS system equipped with an ES ionization chamber. A Waters Symmetry column, C18 5 μm 2.1×30 mm, UV=220 nm, 10 min gradient of 10-90% MeCN/H₂O+0.1% TFA and flow=0.6 mL/min was used. The diastereomeric mixture was used without further purification.

Rₜ=4.54 min. ES-MS m/z: 643.3 [MH⁺]
Rₜ=4.67 min. ES-MS m/z: 643.3 [MH⁺]

### d) Methyl 1-acetyl-L-prolyl-L-leucylglycyl-α-R,S-(4-nitrophenylamino)-glycyl-L-leucyl-β-alaninate.

Methyl 1-acetyl-L-prolyl-L-leucylglycyl-α-R,S-(ethylthio)-glycyl-L-leucyl-β-alaninate (500 mg; 0.78 mmol) and 4-Nitroaniline (215 mg; 1.56 mmol) was dissolved in DCM (20 mL) and THF (15 mL) to give a yellow opaque solution. A molecular sieves were added and the mixture was stirred at room temperature under a protective atmosphere of Argon for 60 min. N-Iodosuccinimide (197 mg; 0.88 mmol) and TfOH (2 μL; 22 μmmol) was added. The reaction was stirred at room temperature for 2.5 hours.

The reaction was quenched using 10% Na₂S₂O₃ (aq) (15 mL) and transferred to a separation funnel using DCM (30 mL). Brine was added and the lower yellow organic phase was separated. The waterphase was extracted with DCM until colourless. The combined yellow organic solutions was dried over Na₂SO₄, filtered and evaporated.

The residual material was dissolved in a small amount of DCM and added onto a short Si-60 gel column, impurities—including unreacted 4-Nitroaniline—were washed out using EtOAc, the product was then washed out with EtOAc/MeOH=5/3 and MeOH. Evaporation of solvent gave a yellow solid. This material was again filtered through a short Si-60 gel column (as described above).

Obtained 339 mg of crude product as a mixture of two diastereomers.

TLC (Si-60, DCM/IPA=9/1) two elongated yellow spots with R_{F}=0.48 and R_{F}=0.32. This product is unstable in acidic conditions.

### e) Methyl 1-acetyl-L-prolyl-L-leucylglycyl-α-R-(4-nitrophenylamino)-glycyl-L-leucyl-β-alaninate

The crude diastereomeric mixture of Methyl 1-acetyl-L-prolyl-L-leucylglycyl-α-R,S-(4-nitrophenylamino)-glycyl-L-leucyl-β-alaninate (339 mg) was separated and further purified using HPLC system A.

The “faster” diastereomer was collected in fractions between 39-45 min and the “slower” diastereomer was collected in fractions between 54-62 min.

Evaporation of MeOH and freeze drying of the water residue gave the title compound and its diastereomer as yellow powder.

The products are unstable in acidic conditions

The “faster” eluting diastereomer was assumed to have “L-like” R-stereochemistry since it was rapidly cleaved by MMPs to liberate 4-Nitroaniline, whereas the “slow” diastereomer was not.

127 mg (22%) of the “fast” L-like diastereomer, title compound was obtained.

HPLC system B: Rₜ=16.07 min. >99% purity.

TLC (Si-60, DCM/IPA=9/1): R_{F}=0.31

¹H-NMR (CD₃CN): δ 8.06 (2H, d), 7.68 (1H, d), 7.65 (1H, t), 7.36 (1H, d), 7.13 (1H, d), 6.92 (1H, brt), 6.79 (2H, d), 6.41 (1H, d), 5.69 (1H, t), 4.25 (1H, q), 4.11 (1H, dt), 4.02 (1H, dd), 3.89 (1H, dd), 3.67 (1H, dd), 3.62 (1H, m), 3.61 (3H, s), 3.49 (1H, m), 3.37 (2H, m), 2.47 (2H, t), 2.11 (1H, m), 2.03 (3H, s), 1.96-1.83 (3H, m), 1.68-1.50 (6H, m), 0.93-0.84 (12H, m) ppm.

¹³C-NMR (CD₃CN): δ 174.63, 173.86, 172.84, 172.45, 172.34, 171.36, 168.29, 152.11, 139.70, 126.62, 113.44, 62.07, 61.27, 53.99, 53.39, 52.10, 49.23, 43.69, 41.50, 39.71, 35.93, 34.48, 30.20, 25.65, 25.56, 25.32, 23.41, 23.20, 22.96, 21.84, 21.39 ppm

95 mg (17%) of the “slow” D-like diastereomer was obtained.

HPLC system B: Rₜ=16.98 min. >99% purity.

TLC (Si-60, DCM:IPA=9:1): R_{F}=0.46

¹H-NMR (CD₃CN): δ 8.06 (2H, d), 7.78 (1H, d), 7.69 (1H, brt), 7.63 (1H, d), 7.22 (1H, d), 7.02 (1H, brt), 6.81 (2H, d), 6.47 (1H, d), 5.80 (1H, t), 4.28-4.17 (3H, m), 3.75 (2H, d), 3.65 (3H, s), 3.63 (1H, m), 3.52 (1H, m), 3.46 (1H, m), 3.34 (1H, m), 2.52 (2H, dt), 2.20 (1H, m), 1.97 (2H, m), 1.94 (1H, m), 1.89 (3H, s), 1.82-1.50 (6H, m), 0.98-0.82 (12H, m) ppm

¹³C-NMR (CD₃CN): δ 175.00, 174.90, 173.06, 172.91, 172.68, 171.24, 168.71, 152.56, 139.63, 126.84, 113.21, 62.18, 60.72, 53.81, 52.85, 52.25, 49.36, 44.73, 41.19, 38.94, 36.18, 34.48, 30.54, 25.72, 25.66, 25.60, 23.46, 23.38, 22.81, 21.63, 21.58 ppm.

### Example 1.2

### Preparation of the Substrate: Methyl 1-acetyl-L-prolyl-L-leucylglycyl-α-R-(4-nitrophenylamino)-glycyl-L-leucylglycinate

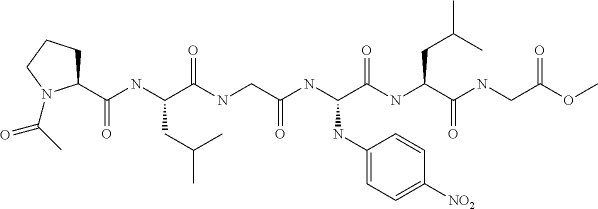

### a) Methyl N-[(9H-fluoren-9-ylmethoxy)carbonyl]glycyl-α-R,S-(ethylthio)-glycyl-L-leucylglycinate

N-[(9H-fluoren-9-ylmethoxy)carbonyl]glycyl-α-R,S-(ethylthio)-glycine (2.09 g; 5.04 mmol) was dissolved in DMF (75 ml), HBTU (2.87 g; 7.57 mmol) and DIEA (0.9 ml; 5.26 mmol) was added. To this solution was added L-leucyl-glycine methyl ester hydrochloride (1.37 g; 5.74 mmol) and additional DIEA (1.9 ml; 11.1 mmol). The slightly yellow mixture was stirred at room temp. for 5 hours, the red reaction mixture was evaporated to remove DMF, the resulting red oil was dissolved in EtOAc and washed with 5% KHSO₄, brine, 5% NaHCO₃ and brine before drying over Na₂SO₄.

After filtration and evaporation the orange oily crude product was purified using flash chromatography on a Si-60 gel with EtOAc:Heptane (5:2) as eluent. The subtitle compound was collected as a slightly yellow oil, that when dissolved in ether and evaporated gave a foam that solidified into a slightly yellow powder, yield 2.31 g (77%) as a 1:1 mixture of the two possible diastereomers.

TLC Si-60, EtOAc:Heptane (5:1) R_{f}=0.37+0.39

¹H-NMR (CDCl₃): δ 8.59+8.46 (tot 1H, d+d), 7.90+7.83 (tot 1H, brt+brt), 7.75-7.70 (3H, brd), 7.59+7.56 (tot 2H, d+d), 7.37 (2H, t), 7.28 (2H, m), 6.61+6.33 (tot 1H, brt+brt), 5.99+5.91 (tot 1H, d+d), 4.90-4.75 (1H, m), 4.46-3.76 (7H, m), 3.54+3.50 (tot 3H, s+s), 2.83-2.59 (2H, m), 1.80-1.60 (3H, m), 1.30-1.16 (3H, m), 0.98-0.88 (6H, m) ppm.

¹³C-NMR (CDCl₃): δ 172.49+172.16, 169.88+169.86, 168.78+168.70, 168.20+168.09, 156.78+156.70, 143.95+143.84+143.70+143.62, 141.18+141.17+141.13, 127.67, 127.03, 125.16+125.12+125.08, 119.90, 67.33, 54.46+54.25, 52.06, 51.90+51.60, 46.98, 44.33+44.25, 42.25+42.13, 41.05+40.95, 24.70+24.57, 24.29+23.77, 23.02+22.63+22.46+21.99, 14.35+14.27 ppm.

### b) Methyl glycyl-α-R,S-(ethylthio)-glycyl-L-leucylglycinate hydrochloride

Methyl N-[(9H-fluoren-9-ylmethoxy)carbonyl]glycyl-α-R,S-(ethylthio)-glycyl-L-leucylglycinate (2.80 g; 4.68 mmol) was dissolved in DMF (100 ml) and 20% piperidine/DMF solution (60 ml) was added. The reaction was followed on TLC Si-60, EtOAc:Heptane (4:1) and after 20 min the reaction was complete. The reaction mixture was evaporated, the residue was dissolved in a minimum of DCM and filtered through a Si-60 gel using more DCM to wash away the fast moving impurities, the product as a free base was then extracted from the silica gel using MeOH as solvent. The product was then treated with HCl/ether to give the subtitle compound in quantitative yield.

The slightly yellow product is hygroscopic and becomes sticky in contact with air.

¹H-NMR (pyridine-d₅): δ 10.47+10.20 (tot 1H, d+d), 10.12+9.92 (tot 1H, d+d), 9.91+9.75 (tot 1H, t+t), 9.55-9.00 (3H, brs), 6.46+6.42 (tot 1H; d+d), 5.27-5.16 (1H, m), 4.76+4.73+4.61 (tot 2H, s+d+d), 4.36-4.14 (tot 2H, m), 3.48 (3H, s), 2.95-2.74 (2H, m), 2.12-1.92 (3H, m), 1.14+1.09 (tot 3H, t+t), 0.94+0.85+0.83 (tot 6H, d+d+d) ppm.

¹³C-NMR (pyridine-d₅): δ 173.60+173.44, 171.03+170.91, 168.79+168.69, 167.50+167.25, 55.63+55.39, 52.97+52.82, 51.77+51.74, 42.13+41.87, 41.99+41.49, 41.81+41.38, 25.12+25.08, 24.79+24.49, 23.33+23.13, 22.01+21.72, 14.76+14.64 ppm.

### c) Methyl 1-acetyl-L-prolyl-L-leucylglycyl-α-R,S-(ethylthio)-glycyl-L-leucylglycinate

Methyl glycyl-α-R,S-(ethylthio)-glycyl-L-leucylglycinate hydrochloride (106 mg; 0.26 mmol), N-acetyl-L-prolyl-L-Leucin (74 mg; 0.27 mmol) and HBTU (159 mg; 0.42 mmol) was dissolved in DMF (5 ml). To the stirred slightly yellow solution was added DIEA (0.14 ml; 0.82 mmol), the reaction was allowed to proceed at room temperature for 21 hours. The dark red solution was evaporated to remove solvent, the red oily residue was dissolved in DCM (50 ml) and washed with 5% KHSO₄ (3×40 ml), brine (50 ml), 5% NaHCO₃ (3×40 ml) and brine (50 ml), dried over MgSO₄, filtration and evaporation gave a red solid crude product (103 mg). This was purified with flash chromatograpy on a Si-60 gel using, DCM:IPA (10:1) as eluent until the fast moving impurities was collected, then the eluent was changed to DCM:IPA (10:2). The fractions containing the product was collected and evaporated to give the subtitle compound (77 mg, 47%) as a slightly yellow powder.

Analytical HPLC system: column Kromasil 100-5C18, 150×4.6 mm, flow 1.0 ml/min., 100% H₂O:MeOH 80:20 (0-0.5 min), gradient to 100% MeOH (0.5-6 min), 100% MeOH (6-7 min.), UV=220 nm.

The diastereomers have Rₜ=6.40 and 6.62 min.

¹H-NMR (pyridine-D5): δ 9.98-9.10 (5H, m), 6.36+6.22 (tot 1H, d+d), 5.15-5.04 (1H, m), 5.04-4.90 (1H, m), 4.75-4.58 (1H, m), 4.50-4.00 (4H, m) 3.55+3.54 (tot 3H, s+s), 3.51-3.39+3.29-3.16 (1H+1H, m+m), 2.91-2.70 (2H, m), 2.35-1.52 (10H, m), 1.91 (3H, s), 1.20-1.02 (3H, m), 0.92-0.62 (12H, m) ppm.

¹³C-NMR (pyridine-D5): δ 173.88, 173.85, 173.34, 173.49, 173.29, 173.24, 170.96, 170.84, 170.46, 170.33, 169.74, 169.44, 168.93, 168.79, 60.92, 60.85, 55.47, 55.22, 52.59, 52.51, 52.42, 52.40, 51.80, 51.79, 48.42, 48.38, 43.54, 43.50, 41.76, 41.75, 41.48, 41.48, 40.84, 40.77, 29.88, 29.82, 25.21, 25.21, 25.09, 25.05, 24.95, 24.94, 24.71, 24.38, 23.23, 23.19, 23.16, 22.99, 22.53, 22.50, 21.91, 21.63, 21.60, 21.57, 14.78, 14.78 ppm.

### d) Methyl 1-acetyl-L-prolyl-L-leucylglycyl-α-R-(4-nitrophenylamino)-glycyl-L-leucylglycinate

The diastereomeric mixture of Methyl 1-acetyl-L-prolyl-L-leucylglycyl-α-R,S-(ethylthio)-glycyl-L-leucylglycinate (0.10 g; 0.16 mmol) was dissolved in DCM (9 ml) and DMF (1 ml) to give a slightly yellow solution. p-Nitroaniline (0.03 g; 0.24 mmol) and crushed 3 A molecular sieves were added. The yellow solution was stirred at room temperature for two hours under nitrogen. N-Iodosuccinimide (0.05 g; 0.21 mmol) dissolved in dry THF (1 ml) was added and directly followed by addition of Trifluoromethanesulfonicacid (2 μl; 22 μmol). The reaction was followed on TLC, Si-60, DCM:IPA (10:1). After 3 hours the red reaction mixture was filtered to remove the molecular sieves, the filtercake was washed with DCM and MeOH before the solvent was evaporated, the residue was dissolved in DCM and washed with 10% Na₂S₂O₃ (20 ml), the yellow phases was separated, the yellow water phase was back extracted once with DCM (10 ml). The combined organic phases was washed with brine and dried (MgSO₄), filtration and evaporation of solvents gave a yellow crude product. Purification and separation of the diastereomers was made using the following procedure. The crude product was first purified by flash chromatography on Si-60 gel using DCM:IPA (10:1) as eluent, the two diastereomers was separated. The fractions containing the products was then further purified using reversed phase HPLC system D, the diastereomers was collected at 12.58 min and 15.17 min.

Evaporation of MeOH and freeze drying of the water residue gave the title compound and its diastereomer as yellow powder.

The products are unstable to acidic conditions

The “faster” eluting diastereomer was assumed to have “L-like” R-stereochemistry since it was rapidly cleaved by MMPs to liberate 4-Nitroaniline, whereas the “slow” diastereomer was not.

10 mg (9%) of the “fast” L-like diastereomer, title compound was obtained.

HPLC system D: Rt=12.58 min.

TLC (Si-60, DCM:IPA=10:1): R_{F}=0.27

¹H-NMR (pyridine-D5): δ 9.89 (1H, d), 9.57 (1H, t), 9.48 (1H, d), 9.24 (1H, d), 9.23 (1H, t), 8.19 (1H, d), 8.10 (2H, d), 6.97 (2H, d), 6.44 (1H, t), 5.16-5.06 (1H, m), 4.95-4.88 (1H, m), 4.65-4.59 (1H, m), 4.43-4.10 (4H, m), 3.55 (3H, s), 3.50-3.43+3.26-3.18 (1H+1H, m+m), 2.30-1.55 (10H, m), 1.90 (3H, s), 0.85-0.72 (12H, m) ppm.

¹³C-NMR (pyridine-D5): δ 173.82, 173.58, 173.33, 171.02, 170.93, 170.66, 168.76, 152.54, 138.89, 126.31, 112.91, 61.14, 61.02, 52.78, 52.74, 51.81, 48.45, 43.45, 41.47, 41.42, 40.29, 29.94, 25.23, 25.09, 24.96, 23.16, 23.14, 22.52, 21.77, 21.49 ppm.

18 mg (16%) of the “slow” D-like diastereomer was obtained.

HPLC system D: Rt=15.17 min.

TLC (Si-60, DCM:IPA=10:1): R_{F}=0.48

¹H-NMR (pyridine-D5): δ 9.50 (1H, d), 9.34 (1H, t), 9.22 (1H, d), 9.18 (1H, t), 9.05 (1H, d), 8.18 (1H, d), 8.11 (2H, d), 7.04 (2H, d), 6.63 (1H, t), 5.09-5.00 (1H, m), 4.89-4.80 (1H, m), 4.68-4.61 (1H, m), 4.41-4.32+4.25-4.13 (2H+2H, m+m), 3.58 (3H, s), 3.54-3.46+3.29-3.20 (1H+1H, m+m), 2.22-1.66 (10H, m), 1.91 (3H, s), 0.88-0.74 (12H, m) ppm.

¹³C-NMR (pyridine-D5): δ 174.36, 173.61, 173.48, 170.99 (2C), 170.86, 168.99, 152.71, 138.95, 126.38, 112.93, 61.04, 60.82, 52.99, 52.33, 51.84, 48.47, 43.95, 41.59, 41.26, 39.82, 29.85, 25.19, 25.10, 25.00, 23.12 (2C), 22.42, 21.68, 21.56 ppm.

### Example 1.3

### Preparation of the substrate: Methyl 1-acetyl-L-prolyl-L-leucylglycyl-α-S-(biphenyl-4-ylmethoxy)-glycyl-L-leucylglycinate

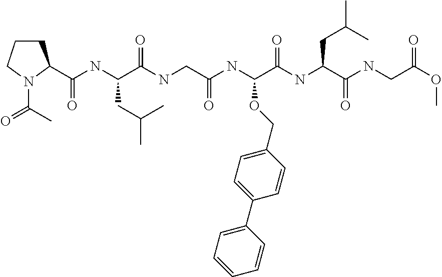

The diastereomeric mixture of Methyl 1-acetyl-L-prolyl-L-leucylglycyl-α-R,S-(ethylthio)-glycyl-L-leucylglycinate (30 mg; 0.048 mmol) was dissolved in DCM (2 mL) and DMF (0.3 mL). Biphenyl-4yl methanol (14.5 mg; 0.079 mmol) and crushed 3 A molecular sieves were added. The slurry was stirred at room temperature for 40 minutes under nitrogen. N-Iodosuccinimide (12 mg; 0.053 mmol) dissolved in dry THF (0.2 mL) was added followed by the addition of Trifluoromethanesulfonicacid (0.001 mL; 0.011 mmol). The brown red slurry was stirred at room temperature for 3.5 hours.

The reaction was quenched by the addition of 10% Na₂S₂O₃ (2 mL), the colourless mixture was filtered and diluted with additional DCM (2 mL) and the organic phase was separated. The waterphase was extracted with DCM (2×2 mL) and the combined organic phases was dried over 3 Å molecular sieves, filtered and evaporated to give a crude product. Purification was made using HPLC system E.

The products are unstable to acidic conditions

The “slower” eluting diastereomer was assumed to have “L-like” R-stereochemistry since it was rapidly cleaved by MMPs to liberate Biphenyl-4yl methanol, whereas the “fast” diastereomer was not.

6.5 mg of the of the “slow” L-like diastereomer, title compound was obtained.

HPLC system F: Rt=4.64 min.

¹H-NMR (pyridine-D5): δ 9.82 (1H, d), 9.46 (1H, t), 9.35 (1H, d), 9.33 (1H, t), 9.09 (1H, d), 7.65-7.47 (6H, m), 7.43 (2H, t), 7.33 (1H, t), 6.31 (1H, d), 5.12 (1H, m), 4.95 (1H, m), 5.00+4.85 (1H+1H, d+d), 4.69 (1H, dd), 4.45 (2H, ddd), 4.31+4.12 (1H+1H, dd+dd), 3.53 (3H, s), 3.49+3.22 (1H+1H, m+m), 2.33-1.54 (10H, m), 1.95 (3H, s), 0.88-0.70 (12H, m) ppm.

10 mg of the “fast” D-like diastereomer was obtained.

HPLC system F: Rt=4.30 min.

¹H-NMR (pyridine-D5): δ 9.88 (1H, d), 9.66 (1H, t), 9.25 (1H, d), 9.15 (1H, t), 9.12 (1H, d), 7.65-7.50 (6H, m), 7.43 (2H, t), 7.33 (1H, t), 6.30 (1H, d), 5.13 (1H, m), 5.01 (1H, m), 4.99+4.84 (1H+1H, d+d), 4.71 (1H, dd), 4.47+4.32 (1H+1H, dd+dd), 4.37+4.18 (1H+1H, dd+dd), 3.54 (3H, s), 3.46+3.21 (1H+1H, m+m), 2.30-1.60 (10H, m), 1.92 (3H, s), 0.88-0.70 (12H, m) ppm.

### Example 1.4

### Preparation of the Substrate: Methyl 1-acetyl-L-prolyl-L-leucylglycyl-α-R-[4-(5-p-tolyl-[1,3,4]oxadiazol-2-yl)-phenylamino]-glycyl-L-leucylglycinate

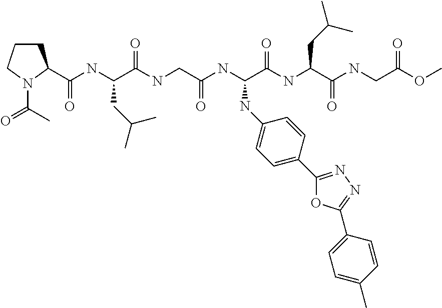

Following the procedure described in Example 1.3 gave a crude product that was further purified as follows. The crude diastereomeric mixture was dissolved in DCM and added onto a Si-60 column, EtOAc was then used to wash out fast moving impurities including unreacted 4-(5-p-tolyl-[1,3,4]oxadiazol-2-yl)-phenylamine. The product was then eluted with EtOAc:MeOH=5:1, TLC (Si-60) Rf=0.27.

TLC (Si-60, DCM:IPA=8:1) Rf=0.3+0.5

The separation of diastereomers was made using HPLC system G.

The faster eluting diastereomer was collected at 12.75 min.

The slower eluting diastereomer was collected at 15.22 min.

Evaporation of MeOH and freeze drying of the water residue gave the title compound and its diastereomer as colourless powder.

The products are unstable to acidic conditions

The “faster” eluting diastereomer was assumed to have “L-like” R-stereochemistry since it was rapidly cleaved by MMPs to liberate 4-(5-p-tolyl-[1,3,4]oxadiazol-2-yl)-phenylamine, whereas the “slow” diastereomer was not.

14.4 mg (36%) of the “fast” L-like diastereomer, title compound was obtained.

HPLC system H: Rt=2.64 min.

TLC (Si-60, DCM:IPA=8:1): R_{F}=0.3

¹H-NMR (pyridine-D5): δ 9.85 (1H, d), 9.55 (1H, t), 9.40 (1H, d), 9.24 (1H, t), 9.23 (1H, d), 8.10 (2H, d), 8.02 (2H, d), 7.48 (1H, d), 7.28 (2H, d), 7.12 (2H, d), 6.47 (1H, t), 5.13 (1H, m), 4.94 (1H, m), 4.63 (1H, dd), 4.39 (2H, d), 4.35+4.18 (1H+1H, dd+dd), 3.56 (3H, s), 3.46+3.22 (1H+1H, m+m), 2.24 (3H, s), 1.92 (3H, s), 2.20-1.65 (10H, m), 0.88-0.70 (12H, m) ppm.

3.1 mg (8%) of the “slow” D-like diastereomer was obtained.

HPLC system H: Rt=3.21 min.

TLC (Si-60, DCM:IPA=8:1): R_{F}=0.5

¹H-NMR (pyridine-D5): δ 9.46 (1H, d), 9.27 (1H, t), 9.25 (1H, d), 9.20 (1H, t), 9.05 (1H, d), 8.11 (2H, d), 8.04 (2H, d), 7.51 (1H, d), 7.28 (2H, d), 7.21 (2H, d), 6.65 (1H, t), 5.09 (1H, m), 4.85 (1H, m), 4.67 (1H, dd), 4.39+4.24 (1H+1H, dd+dd), 4.38+4.19 (1H+1H, dd+dd), 3.59 (3H, s), 3.50+3.25 (1H+1H, m+m), 2.24 (3H, s), 1.93 (3H, s), 2.22-1.67 (10H, m), 0.87-0.76 (12H, m) ppm.

### Example 15

### Preparation of the Substrate: 1-acetyl-L-prolyl-L-leucylglycyl-α-R-(4-nitrophenylamino)-glycyl-N-phenyl-L-phenylalaninamide

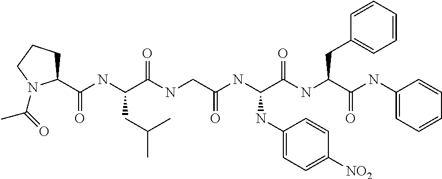

### a) 1-acetyl-L-prolyl-L-leucylglycyl-α-R,S-(ethylthio)-glycyl-N-phenyl-L-phenylalaninamide

Following the procedure outlined in Example 1.2a-c, but substituting the N-terminal amino acid in step 1.2a with N-phenyl-L-phenylalaninamide, the diastereomeric mixture of 1-acetyl-L-prolyl-L-leucylglycyl-α-R,S-(ethylthio)-glycyl-N-phenyl-L-phenylalaninamide was synthesised.

HPLC system I: Rt=18.9 min.

ES-MS m/z: 667.4 [MH⁺]

### b) 1-acetyl-L-prolyl-L-leucylglycyl-α-R-(4-nitrophenylamino)-glycyl-N-phenyl-L-phenylalaninamide

Reaction of compound 1.5a with 4-nitroaniline was made as described in Example 1.2d. The crude product containing the diastereomeric mixture was purified and separated using HPLC system J. Evaporation of MeCN and freeze drying of the water residue gave the title compound and its diastereomer as yellow powder.

The products are unstable to acidic conditions

The “faster” eluting diastereomer was assumed to have “L-like” R-stereochemistry since it was rapidly cleaved by MMPs to liberate 4-nitroaniline, whereas the “slow” diastereomer was not.

The “fast” L-like diastereomer, title compound.

HPLC system K: Rt=23.20 min.

¹H-NMR (pyridine-D5): δ 11.02 (1H, s), 9.95 (1H, d), 9.74 (1H, d), 9.24 (1H, d), 9.23 (1H, t), 8.28 (1H, d), 8.08 (2H, d), 7.92 (2H, d), 7.36-6.90 (10H, m), 6.49 (1H, t), 5.29 (1H, m), 4.98 (1H, m), 4.63 (1H, dd), 4.39+4.31 (1H+1H, dd+dd), 3.48+3.30 (1H+1H, m+m), 3.45+3.21 (1H+1H, m+m), 1.88 (3H, s), 2.20-1.65 (7H, m), 0.81 (3H, d), 0.72 (3H, d) ppm.

¹³C-NMR (pyridine-D5): δ 173.96, 173.52, 171.11, 170.66, 170.50, 168.67, 152.59, 139.74, 138.93, 137.86, 129.81, 129.19, 128.80, 127.03, 126.32, 124.21, 120.59, 112.91, 61.14, 61.03, 56.62, 52.72, 48.46, 43.39, 40.39, 38.67, 29.94, 25.23, 25.09, 23.14, 22.51, 21.48 ppm.

The “slow” D-like diastereomer.

HPLC system K: Rt=23.96 min.

¹H-NMR (pyridine-D5): δ 11.05 (1H, s), 9.41 (1H, d), 9.16 (2H, d+t), 8.94 (1H, d), 8.10 (3H, m), 7.89 (2H, d), 7.40-7.08 (8H, m), 6.99 (2H, d), 6.48 (1H, t), 5.20 (1H, m), 4.92 (1H, m), 4.62 (1H, dd), 4.37+4.18 (1H+1H, dd+dd), 3.55+3.37 (1H+1H, dd+dd), 3.35+3.13 (1H+1H, m+m), 1.84 (3H, s), 2.20-1.63 (7H, m), 0.85 (3H, d), 0.83 (3H, d) ppm.

¹³C-NMR (pyridine-D5): δ 174.70, 173.67, 171.23, 171.01, 170.91, 168.95, 152.80, 139.58, 138.89, 137.99, 129.80, 129.20, 128.85, 127.11, 126.42, 124.38, 120.73, 112.77, 61.15, 60.68, 57.16, 52.13, 48.40, 44.28, 39.53, 38.48, 30.01, 25.12(2C), 23.19, 22.32, 21.64 ppm.

### Example 2

### Biochemical Substrate Activity with Purified Proteases

In order to confirm the specificity of the substrates for various MMPs, standard enzymatic assays were adapted to monitor the release of reporter. The reporter was either analysed by a LC-MS method as described in Example 3a, or by a LC-UV method as described in Example 4.

### MMP8

Purified pro-MMP8 is purchased from Calbiochem. The enzyme (at 10 μg/ml) is activated by p-amino-phenyl-mercuric acetate (APMA) at 1 mM for 2.5 h, 35° C. The activated enzyme can be used to monitor activity of substrates as follows: MMP8 (200 ng/ml final concentration) is incubated for 90 minutes at 35° C. (80% H2O) with one of the substrates of Example 1 (12.5 μM) in assay buffer (0.1M “Tris-HCl” (trade mark) buffer, pH 7.5 containing 0.1M NaCl, 30 mM CaCl₂, 0.040 mM ZnCl and 0.05% (w/v) “Brij 35” (trade mark) detergent) in the presence or absence of inhibitors. Activity is determined by measuring the release of reporter 4-nitroaniline by the using the method in Example 3a.

### MMP9

Recombinant human MMP9 catalytic domain was expressed and then purified by Zn chelate column chromatography followed by hydroxamate affinity column chromatography. The enzyme can be used to monitor activity of substrates as follows: MMP9 (5 ng/ml final concentration) is incubated for 30 minutes at room temperature (RT) with one of the substrates of Example 1 (5 μM) in assay buffer (0.1M “Tris-HCl” (trade mark) buffer, pH 7.3 containing 0.1M NaCl, 20 mM CaCl₂, 0.020 mM ZnCl and 0.05% (w/v) “Brij 35” (trade mark) detergent) in the presence or absence of inhibitors. Activity is determined by measuring the release of reporter 4-nitroaniline by the using the method in Example 3a.

### MMP12

Recombinant human MMP12 catalytic domain may be expressed and purified as described by Parkar A. A. et al, (2000), Protein Expression and Purification, 20, 152. The purified enzyme can be used to monitor activity of substrates as follows: MMP12 (50 ng/ml final concentration) is incubated for 60 minutes at room temperature with one of the substrates of Example 1 (10 μM) in assay buffer (0.1M “Tris-HCl” (trade mark) buffer, pH 7.3 containing 0.1M NaCl, 20 mM CaCl₂, 0.020 mM ZnCl and 0.05% (w/v) “Brij 35” (trade mark) detergent) in the presence or absence of inhibitors. Activity is determined by measuring the release of reporter 4-nitroaniline by the using the method in Example 3a.

### MMP14

Recombinant human MMP14 catalytic domain may be expressed and purified as described by Parkar A. A. et al, (2000), Protein Expression and Purification, 20, 152. The purified enzyme can be used to monitor activity of substrates as follows: MMP14 (10 ng/ml final concentration) is incubated for 60 minutes at room temperature with one of the substrates of Example 1 (10 μM) in assay buffer (0.1M “Tris-HCl” (trade mark) buffer, pH 7.5 containing 0.1M NaCl, 20 mM CaCl₂, 0.020 mM ZnCl and 0.05% (w/v) “Brij 35” (trade mark) detergent) in the presence or absence of inhibitors. Activity is determined by measuring the release of reporter 4-nitroaniline by the using the method in Example 3a.

### MMP19

Recombinant human MMP19 catalytic domain may be expressed and purified as described by Parkar A. A. et al, (2000), Protein Expression and Purification, 20:152. The purified enzyme can be used to monitor activity of substrates as follows: MMP19 (40 ng/ml final concentration) is incubated for 120 minutes at 35° C. with one of the substrates of Example 1 (5 μM) in assay buffer (0.1M “Tris-HCl” (trade mark) buffer, pH 7.3 containing 0.1M NaCl, 20 mM CaCl₂, 0.020 mM ZnCl and 0.05% (w/v) “Brij 35” (trade mark) detergent) in the presence or absence of inhibitors. Activity is determined by measuring the release of reporter 4-nitroaniline by the using the method in Example 3a.

A protocol for testing against other matrix metalloproteinases, including MMP9, using expressed and purified pro MMP is described, for instance, by C. Graham Knight et al., (1992) FEBS Lett., 296(3), 263-266.

TABLE 1a
Activity of human MMPs on 30 μM of Methyl 1-acetyl-L-prolyl-L-
leucylglycyl-α-R-(4-nitrophenylamino)-glycyl-L-leucyl-β-alaninate
(Ex. 1.1)

| | MMP Conc. | | |
|---|---|---|---|
| MMP | (nM) | Source | Activity (units/s) |
| 1 | 9.3 | Calbiochem #444208 | 0.16 |
| 2 | 0.71 | Chemicon #CC071 | 2.3 |
| 3 | 19 | AZL0095* | 0.048 |
| 8 | 2.7 | Calbiochem #444229 | 1.1 |
| 9 | 0.27 | AZL0064* | 7.4 |
| 12 | 0.27 | ABL010827* | 7.7 |
| 13 | 1.2 | AZAP0.5 mg/ml* | 1.9 |
| 14 | 4.8 | AZL0078* | 1.0 |
| 16 | 4.3 | Calbiochem #475939 | 1.4 |
| 19 | 19 | AZL0095* | 0.13 |

Enzyme sources denoted with * were produced in-house by AstraZeneca.

TABLE 1b
Activity of selected substrates with MMPs
Activity, units/s (mol_{RXH}/mol_{Enz} * s)

| | Example 1.4 | Example 1.1 | Example 1.5 | Example 1.3 |
|---|---|---|---|---|
| MMP1 | 0.00 | 26.1 | 2.48 | 35.4 |
| MMP3 | 0.00 | 3.8 | 0.18 | 5.5 |
| MMP8 | 0.05 | 120.4 | 11.4 | 26.9 |
| MMP9 | 0.58 | 62.9 | 11.5 | 63.6 |

### Example-3a

### Determination of 4-Nitroaniline in Plasma Using LC-MS/MS

### Preparation of the Sample

Samples of heparinised whole blood (1 mL) were stimulated with Zymosan (0, 50, 300, 600, 900 and 1200 μg/mL), vortexed or and incubated at 37° C. for 15 minutes. Plasma was collected after centrifugation, mixed with the substrate (10 μM) and incubated at 37° C. for 60 minutes. Methanol (300 μL), with (¹³C)₆-4-nitroaniline (1 μM) as internal standard, was added to a 100 μL aliquot of the above mixture and centrifuged to remove precipitated proteins. The supernatant (100 μL) was diluted with water (100 μL) and the extracts was injected directly onto a HPLC column.

### Calibration Curve

A seven point calibration curve is produced by spiking blank plasma with 4-nitroaniline to concentrations between 1.4 and 2976 nM. The calibration samples are then precipitated and diluted according to above method.

Also an unspiked plasma sample is produced and used as a blank sample.

### LC-MS/MS System

The LC-MS system consists of two gradient pumps, one isocratic pump, an autoinjector and a triquadropole masspectrometer (Sciex API3000). The samples are injected onto a precolumn (NH₂, 2×10 mm), which retains the matrix components, which is then back flushed with ethanol. The final elution is done by a gradient (C₁₈-column, 2×40 mm) from 0 to 100% B in 6 minutes followed by equilibration for 1.5 minutes. Mobile phase A: MeOH:50 mM NH₄Ac pH 4.0 (2:98) and B: MeOH:50 mM NH₄Ac pH 4.0 (90:10).

The detection is performed by multiple reaction monitoring (MRM). For 4-nitroaniline the m/z of 139.1 is used with the fragment at 122.2 and for dexamethasone the m/z of 393.1 with the fragment at 373.0.

### Evaluation

The ratio (area 4-nitroaniline/area internal standard) is calculated for all calibration samples and plotted against the concentration. All unknown samples are then calculated from the calibration curve.

### Example 3b

### Determination of MMP9 Activity in Whole Blood Via Analysis of 4-Nitroaniline in Plasma Samples Taken from Patients

The aim of this work was to determine the MMP9 activity in blood samples via analysis of 4-nitroaniline in plasma samples taken from patients with moderate COPD, asymptomatic smokers and healthy volunteers.

Materials & Methods for the Determination of 4-Nitroaniline in Plasma Samples by LC-MS

The following materials were used:

Dimethyl sulphoxide, from Fisher Scientific, Loughborough, UK.
Methanol 205 grade from Romil Ltd., Waterbeach, UK.
HPLC grade acetonitrile, from Fisher Scientific, Loughborough, UK.
Ammonia (0.89, 35%), HPLC grade, from Fisher Scientific, Loughborough, UK.
4-nitroaniline, code number 18, 531-0, Aldrich Chemical Co. Milwaukee, USA.
[¹³C]₆-4-nitroaniline, Medicinal Chemistry AstraZeneca R & D Charnwood. [¹³C]₆-4-nitroaniline can be obtained by nitration and hydrolysis of commercially available ¹³C₆N-Phenylacetamide.
Methyl 1-acetyl-L-prolyl-L-leucylglycyl-α-R-(4-nitrophenylamino)-glycyl-L-leucyl-β-alaninate—produced as described in Example 1.1.
Control human plasma (lithium heparin) from Charterhouse Clinical Research Unit, London, UK.
Water freshly prepared by MilliQ purification system, Millipore, Watford, UK.

Equipment

The following equipment was used

Unicam UV300 series UV/Vis spectrophotometer from Spectronic Unicam, Cambridge, UK.
Sciex API 150ex mass spectrometer with dedicated data system, from Applied Biosystems, Warrington, Cheshire, UK.
HP1100 solvent degasser, HP1100 binary gradient pump, HP1100 thermostatted autosampler and HP1100 column oven from Agilent Technologies Ltd., Altrincham, Cheshire, UK.
Luna phenyl-hexyl HPLC column (5 μm, 50×2.0 mm) (Phenomenex, part no. 00B-4257-B0).
Luna phenyl-propyl HPLC security guard column (4.0×2.0 mm) (Phenomenex, part no. AJO-4350).

The reagents used herein are known in the art.

Preparation of Calibration and Quality Control Samples

### 1. Preparation of Calibration Samples

Using appropriate pipettes, prepare plasma calibration samples of 4-nitroaniline at 0.050, 0.100, 0.200, 0.500, 1.00, 2.00, 5.00, and 10.0 μM as shown in Table 1. Mix thoroughly using a vortex mixer.

TABLE 1
Preparation of plasma calibration standards

| Concentration of | | Volume of Stock | |
|---|---|---|---|
| 4-nitroaniline in | Stock solution or | solution or | Volume of |
| calibration | Calibration | Calibration | Control human |
| sample (μM) | standard | standard (μL) | plasma (μL) |
| 10.0 | B | 50 | 2450 |
| 5.00 | Cal 10.0 | 250 | 250 |
| 2.00 | Cal 10.0 | 100 | 400 |
| 1.00 | Cal 10.0 | 50 | 450 |
| 0.500 | Cal 5.00 | 50 | 450 |
| 0.200 | Cal 2.00 | 50 | 450 |
| 0.100 | Cal 1.00 | 50 | 450 |
| 0.050 | Cal 0.500 | 50 | 450 |

### 2. Preparation of Plasma Quality Control Samples

Using appropriate pipettes, prepare quality control samples at 0.100, 2.00, and 8.00 μM 4-nitroaniline as shown in Table 2. Thoroughly mix by vortex the contents of the vials and dispense aliquots (approximately 300 μL, sufficient for two analyses) of each sample into plastic Sarstedt tubes (2.0 mL). Store the tubes in a freezer at −20° C. or below.

TABLE 2
Preparation of quality control samples

| Concentration | Stock | | |
|---|---|---|---|
| of | solution or | Volume of Stock | Volume of pooled |
| Quality Control | Quality Control | solution or Quality | human plasma |
| sample (μM) | sample | Control sample (μL) | (μL) |
| 8.00 | B | 160 | 9840 |
| 2.00 | QC 8.00 | 2500 | 7500 |
| 0.100 | QC 2.00 | 500 | 9500 |

Sample Analysis Procedures

1. Methyl 1-acetyl-L-prolyl-L-leucylglycyl-α-R-(4-nitrophenylamino)-glycyl-L-leucyl-β-alaninate incubation in plasma

Thaw, as required, pooled control human plasma, quality control and test samples. Add methyl 1-acetyl-L-prolyl-L-leucylglycyl-α-R-(4-nitrophenylamino)-glycyl-L-leucyl-β-alaninate (2 μL, solution D) to test plasma sample (200 μL) in 2 mL screw cap microtubes and mix on a vortex mixer.

Incubate tubes for 60 minutes at 37° C. in a shaking water bath. Prepare the calibration samples in plasma as detailed in Table 1 during the 60 minute incubation of test samples in step 3. Mix the samples on a vortex mixer.

Manually dispense aliquots (100 μL) of each plasma sample into separate wells of a 96 deep-well 2 mL plate using an appropriate pipette. (Note: start pipetting calibration, quality control and blank samples approximately 10 minutes before the test samples have finished their incubation in step 3). Dispense internal standard in methanol (300 μL) into each well using the Tecan. Cover the plate with polystyrene cover. Centrifuge at 3000 rpm for 5 minutes at room temperature.

Transfer an aliquot (100 μL) of the sample supernatant into separate wells of a 96 deep-well 2 mL plate using the Tecan. Mix water (100 μL) with each sample in the wells of a 96 deep-well polypropylene plate using the Tecan. Cover plate with pre-spilt 96 well seal.

Analyse 10 μL aliquots by LC/MS.

LC/MS Analysis of Prepared Extracts

### 1. Equipment —

### PUM HP100

Degasser: HP1100 Autosampler: HP1100 A/S thermostat: HP1100 Column: HP1100 MS: Sciex API 150ex

### 2. HPLC Conditions for Test Samples

Column: Luna phenyl-hexyl HPLC column (5 μm, 50×2.0 mm) with Luna phenyl-propyl HPLC security guard column pre-column (4.0×2.0 mm)
Solvent A: 0.01% aqueous ammonia

### Solvent B: Methanol

### Composition: See Table 3

### Flow: See Table 3

### Column Temperature: 40° C.

Run time: 10 minutes
Injection volume: 10 μL
Needle wash: 25% acetonitrile/water for 3 seconds

TABLE 3
HPLC gradient conditions

| Time | Flow | | | | |
|---|---|---|---|---|---|
| (min) | (μL/min) | % A | % B | TE # 1 | TE # 2 |
| 0 | 300 | 60 | 40 | | |
| 1.0 | 300 | 10 | 90 | close | close |
| 4.0 | 300 | 10 | 90 | | |
| 4.5 | 600 | 10 | 90 | | |
| 5.0 | 600 | 60 | 40 | | |
| 10 | 300 | 60 | 40 | | |

The HPLC eluant is directed into the mass spectrometer between 1 and 4 minutes. For the remainder of the run it is directed to waste.

### 3. MS Acquisition

The HPLC column effluent is directed into the mass spectrometer via a heated nebuliser interface, in negative mode. 4-nitroaniline and [¹³C]₆-4-nitroaniline are detected using single ion monitoring of the [M-H]⁻ ions at the mass to charge ratio of m/z 137 and 143 respectively.

### Temperature: 425° C.

Nebuliser gas: 11 (arbitrary scale)
Curtain gas: 10 (arbitrary scale)
Auxiliary gas approx. 1 L/min
Dwell time: 500 ms
Resolution: approx. 1 amu
Acquisition time: 5.0 min
Processing software: PE Sciex ‘Analyst’ v1.1

Orifice and ring voltages may be selected as necessary in order to maximise sensitivity.

Samples should be analysed in the following order:

a) Conditioning samples (10 μM×4)
b) Calibration samples (10.0, 2.00, 0.500, 0.100 μM)
c) Quality control samples (0.01, 2.00, 8.00 μM)
d) Blank sample
e) Test samples
f) Quality control samples (0.01, 2.00, 8.00 μM)
g) Blank sample
h) Calibration samples (5.00, 1.00, 0.200, 0.050 μM)

Determine the peak response of the analyte and internal standard in each sample (expected retention time approx. 1.8 min). Apply a suitable curve fit to the data from the calibration samples within the Analyst software to allow quantification of the concentration of 4-nitroaniline present in each sample.

Standard Procedure for Data

Determine the peak area of the analyte in each sample and apply an appropriate linear regression procedure to the data from the calibration samples.

### Experiment

Human plasma samples were analysed in 12 analysis batches using a validated protein precipitation LC-MS method. The analysis batches included test samples, calibration samples and duplicate quality control samples. All 12 batches were acceptable, however 1 result was rejected due to incorrect sample preparation.

Whole blood samples were stimulated with varying concentrations of zymosan (0, 50, 300, 600, 900 and 1200 μg/mL). Plasma sample obtained from the stimulated whole blood samples were mixed with the substrate methyl 1-acetyl-L-prolyl-L-leucylglycyl-α-R-(4-nitrophenylamino)-glycyl-L-leucyl-β-alaninate at a plasma concentration of 10 μM and the samples were then incubated at 37° C. for 60 minutes. Aliquots (100 μL) of the human plasma were mixed with methanol (300 μL) containing [¹³C]₆-4-nitroaniline as an internal standard to precipitate the proteins and the sample was centrifuged and supernatants (100 μL) were mixed with water (100 μL). The extracts 10 μL were injected directly onto a Phenomenex Luna phenyl-hexyl column (50×2.0 mm, 5 μm) and the compounds eluted with a gradient mobile phase consisting of 0.01% ammonia/methanol. 4-nitroaniline was resolved from endogenous plasma constituents and the HPLC effluent was directed into the mass spectrometer via a heated nebuliser interface in negative mode. 4-nitroaniline and [¹³C]₆-4-nitroaniline were detected using single ion monitoring of the [M-H]⁻ ions at the mass to charge ratio of m/z 137 and 143 respectively. The method is described above.

The method is validated over the range 0.0500 to 10.0 μM for 4-nitroaniline based on the analysis of a 100 μL plasma sample. Quality control samples at concentrations of 0.100, 2.00 and 8.00 μM 4-nitroaniline were included to determine the acceptability of each analysis batch. The limit of quantification (LOQ) of the method is 0.0500 μM 4-nitroaniline for a 100 μL aliquot of plasma.

### Results

Throughout mean and standard deviation data are presented to 3 significant figures.

### 1. Test Samples

The individual concentrations for the determination of 4-nitroaniline from the healthy volunteers from visits 3 and 4 are shown in Table 4 and Table 5 respectively with the mean values illustrated in FIG. 1. The mean values ranged from 0.121 μM at 0 μg zymosan to 3.59 μM at 1200 μg zymosan for visit 3 and 0.131 μM at 0 μg zymosan to 3.39 μM at 1200 μg zymosan for visit 4.

TABLE 4
Concentration of 4-nitroaniline obtained from healthy volunteers
during visit 3.

| | |
|---|---|
| | 4-nitroaniline concentration (μM) |
| Subject | Zymosan concentration (μg) |

| number | 0 | 50 | 300 | 600 | 900 | 1200 |
|---|---|---|---|---|---|---|
| 101 | 0.118 | 0.646 | 1.56 | 1.84 | 2.07 | 2.14 |
| 102 | 0.123 | 1.16 | 2.28 | 2.95 | 2.99 | 3.02 |
| 103 | 0.145 | 1.13 | 2.48 | 3.00 | 3.05 | 3.07 |
| 104 | 0.132 | 1.82 | 3.68 | 4.26 | 4.50 | 4.53 |
| 105 | 0.126 | 1.13 | 2.65 | 2.98 | 3.14 | 2.99 |
| 106 | 0.0917 | 2.10 | 4.32 | 4.39 | 4.38 | 4.28 |
| 107 | 0.145 | 1.52 | 3.63 | 4.23 | 4.24 | 4.45 |
| 108 | 0.124 | 0.988 | 2.82 | 3.90 | 3.98 | 4.41 |
| 109 | 0.104 | 0.836 | 2.31 | 3.08 | 3.45 | 3.53 |
| 110 | 0.0963 | 0.878 | 2.42 | 2.99 | 3.35 | 3.50 |
| Mean | 0.121 | 1.22 | 2.82 | 3.36 | 3.52 | 3.59 |
| SD | 0.0185 | 0.458 | 0.823 | 0.809 | 0.761 | 0.806 |

TABLE 5
Concentration of 4-nitroaniline obtained from healthy volunteers
during visit 4.

| | |
|---|---|
| | 4-nitroaniline concentration (μM) |
| Subject | Zymosan concentration (μg) |

| number | 0 | 50 | 300 | 600 | 900 | 1200 |
|---|---|---|---|---|---|---|
| 101 | 0.0866 | 0.751 | 1.65 | 1.92 | 2.09 | 2.26 |
| 102 | 0.152 | 1.00 | 2.77 | 2.96 | 3.17 | 3.20 |
| 103 | 0.154 | 1.49 | 2.81 | 3.17 | 3.27 | 3.32 |
| 104 | 0.192 | 2.11 | 4.29 | 4.36 | 5.53 | 5.28 |
| 105 | 0.106 | 0.808 | 1.92 | 2.39 | 2.69 | 2.84 |
| 106 | 0.0977 | 0.616 | 1.68 | 2.12 | 2.46 | 2.85 |
| 107 | 0.130 | 0.732 | 1.98 | 2.41 | 3.03 | 3.15 |
| 108 | 0.199 | 0.795 | 2.29 | AR | 3.48 | 4.00 |
| 109 | 0.0803 | 0.758 | 2.22 | 3.36 | 3.48 | 3.60 |
| 110 | 0.111 | 0.796 | 2.28 | 2.91 | 3.43 | 3.38 |
| Mean | 0.131 | 0.986 | 2.39 | 2.84 | 3.26 | 3.39 |
| SD | 0.0422 | 0.463 | 0.776 | 0.747 | 0.923 | 0.814 |

AR = Analysis rejected

The individual concentrations for the determination of 4-nitroaniline from the smokers from visits 3 and 4 are shown in Table 6 and Table 7 respectively with the mean values illustrated in FIG. 2. The mean values ranged from 0.149 μM at 0 μg zymosan to 4.47 μM at 1200 μg zymosan for visit 3 and 0.142 μM at 0 μg zymosan to 4.41 μM at 1.200 μg zymosan for visit

TABLE 6
Concentration of 4-nitroaniline obtained from smokers during visit 3.

| | |
|---|---|
| | 4-nitroaniline concentration (μM) |
| Subject | Zymosan concentration (μg) |

| number | 0 | 50 | 300 | 600 | 900 | 1200 |
|---|---|---|---|---|---|---|
| 201 | 0.339 | 3.01 | 6.45 | 7.79 | 8.61 | 8.66 |
| 202 | 0.116 | 2.15 | 4.19 | 4.93 | 5.09 | 5.13 |
| 203 | 0.131 | 1.10 | 2.68 | 3.27 | 3.36 | 3.37 |
| 204 | 0.113 | 0.769 | 2.05 | 2.31 | 2.68 | 2.43 |
| 205 | 0.107 | 1.11 | 2.56 | 3.48 | 3.61 | 3.68 |
| 206 | 0.109 | 1.12 | 3.14 | 3.72 | 3.91 | 4.25 |
| 207 | 0.0861 | 0.862 | 2.21 | 2.75 | 3.16 | 3.23 |
| 208 | 0.176 | 3.00 | 6.36 | 7.70 | 8.43 | 8.30 |
| 209 | 0.239 | 1.02 | 2.68 | 3.39 | 3.82 | 4.01 |
| 210 | 0.0779 | 0.406 | 1.18 | 1.70 | 1.82 | 1.64 |
| Mean | 0.149 | 1.45 | 3.35 | 4.10 | 4.45 | 4.47 |
| SD | 0.0817 | 0.928 | 1.78 | 2.10 | 2.31 | 2.32 |

TABLE 7
Concentration of 4-nitroaniline obtained from smokers during visit 4.

| | |
|---|---|
| | 4-nitroaniline concentration (μM) |
| Subject | Zymosan concentration (μg) |

| number | 0 | 50 | 300 | 600 | 900 | 1200 |
|---|---|---|---|---|---|---|
| 201 | 0.279 | 4.57 | 7.93 | 8.74 | 9.12 | 9.95 |
| 202 | 0.131 | 2.16 | 3.57 | 3.92 | 4.48 | 4.81 |
| 203 | 0.165 | 1.45 | 2.65 | 2.87 | 3.10 | 2.95 |
| 204 | 0.0729 | 1.17 | 2.29 | 2.48 | 2.86 | 2.82 |
| 205 | 0.0973 | 1.92 | 3.43 | 3.64 | 4.06 | 4.22 |
| 206 | 0.182 | 1.54 | 2.99 | 3.41 | 3.56 | 3.53 |
| 207 | 0.108 | 0.742 | 2.30 | 2.54 | 3.18 | 3.71 |
| 208 | 0.128 | 1.52 | 4.52 | 5.50 | 6.30 | 6.49 |
| 209 | 0.195 | 1.21 | 2.59 | 3.43 | 3.86 | 4.23 |
| 210 | 0.0588 | 0.341 | 0.91 | 1.1 | 1.38 | 1.42 |
| Mean | 0.142 | 1.66 | 3.32 | 3.76 | 4.19 | 4.41 |
| SD | 0.0657 | 1.15 | 1.88 | 2.08 | 2.14 | 2.36 |

The individual concentrations for the determination of 4-nitroaniline from the COPD patients from visits 3 and 4 are shown in Table 8 and Table 9 respectively with the mean values illustrated in FIG. 3. The mean values ranged from 0.128 μM at 0 μg zymosan to 4.47 μM at 900 μg zymosan for visit 3 and 0.141 μM at 0 μg zymosan to 4.54 μM at 1200 μg zymosan for visit 4.

TABLE 8
Concentration of 4-nitroaniline obtained from COPD patients
during visit 3.

| | |
|---|---|
| | 4-nitroaniline concentration (μM) |
| Subject | Zymosan concentration (μg) |

| number | 0 | 50 | 300 | 600 | 900 | 1200 |
|---|---|---|---|---|---|---|
| 301 | 0.140 | 1.30 | 2.95 | 3.90 | 3.96 | 4.66 |
| 302 | 0.0827 | 1.01 | 3.26 | 4.10 | 4.64 | 4.63 |
| 303 | 0.128 | 1.05 | 2.59 | 2.82 | 3.11 | 3.21 |
| 304 | 0.111 | 2.06 | 4.99 | 5.53 | 6.55 | 6.14 |
| 305 | 0.124 | 2.19 | 4.83 | 5.39 | 5.89 | 5.62 |
| 306 | 0.212 | 1.59 | 3.53 | 4.37 | 4.66 | 4.58 |
| 307 | 0.0914 | 1.37 | 4.29 | 5.06 | 5.97 | 5.89 |
| 308 | 0.162 | 1.20 | 2.10 | 2.57 | 2.88 | 2.58 |
| 309 | 0.139 | 1.89 | 3.29 | 4.58 | 4.97 | 5.06 |
| 310 | 0.138 | 2.48 | 5.21 | 5.75 | 6.29 | 6.10 |
| 311 | 0.114 | 1.05 | 2.27 | 2.92 | 3.33 | 2.67 |
| 312 | 0.137 | 2.00 | 3.73 | 4.74 | 5.36 | 4.38 |
| 313 | 0.137 | 2.89 | 5.39 | 5.89 | 6.13 | 6.04 |
| 314 | 0.115 | 1.55 | 4.39 | 5.53 | 6.16 | 5.92 |
| 315 | 0.0647 | 2.01 | 3.91 | 4.14 | 4.50 | 4.15 |
| 316 | 0.243 | 2.71 | 6.38 | 7.73 | 8.39 | 8.93 |
| 317 | 0.0851 | 0.927 | 2.01 | 2.92 | 3.07 | 3.02 |
| 318 | 0.0527 | 0.281 | 1.17 | 1.82 | 1.82 | 1.95 |
| 319 | 0.118 | 0.969 | 2.52 | 3.46 | 3.95 | 3.88 |
| 320 | 0.165 | 1.00 | 2.41 | 3.45 | 3.83 | 4.14 |
| Mean | 0.128 | 1.58 | 3.56 | 4.33 | 4.77 | 4.68 |
| SD | 0.0453 | 0.682 | 1.35 | 1.42 | 1.59 | 1.63 |

TABLE 9
Concentrations of 4-nitroaniline obtained from COPD patients
during visit 4.

| | |
|---|---|
| | 4-nitroaniline concentration (μM) |
| Subject | Zymosan concentration (μg) |

| number | 0 | 50 | 300 | 600 | 900 | 1200 |
|---|---|---|---|---|---|---|
| 301 | 0.139 | 1.18 | 2.79 | 3.01 | 3.59 | 3.62 |
| 302 | 0.0844 | 1.48 | 3.35 | 3.79 | 4.18 | 4.22 |
| 303 | 0.179 | 1.48 | 3.18 | 3.67 | 4.18 | 3.79 |
| 304 | 0.0951 | 1.25 | 2.95 | 3.49 | 3.70 | 3.85 |
| 305 | 0.138 | 1.88 | 3.95 | 4.75 | 5.05 | 4.85 |
| 306 | 0.243 | 1.84 | 3.77 | 4.63 | 5.26 | 4.91 |
| 307 | 0.110 | 2.11 | 3.61 | 4.45 | 4.58 | 4.43 |
| 308 | 0.217 | 1.34 | 2.63 | 2.99 | 3.30 | 3.47 |
| 309 | 0.125 | 2.16 | 3.71 | 4.23 | 4.42 | 4.87 |
| 310 | 0.115 | 1.58 | 3.74 | 4.58 | 4.90 | 5.22 |
| 311 | 0.143 | 1.20 | 3.01 | 3.55 | 4.06 | 4.06 |
| 312 | 0.125 | 1.67 | 4.12 | 4.62 | 4.94 | 5.42 |
| 313 | 0.134 | 1.18 | 4.14 | 5.28 | 5.13 | 5.50 |
| 314 | 0.185 | 1.44 | 4.77 | 5.90 | 6.15 | 6.49 |
| 315 | 0.0984 | 1.87 | 4.03 | 4.83 | 5.14 | 5.33 |
| 316 | 0.219 | 2.33 | 5.86 | 6.52 | 7.33 | 7.40 |
| 317 | 0.110 | 2.15 | 0.789 | 2.76 | 3.18 | 3.39 |
| 318 | 0.0972 | 0.675 | 1.95 | 2.62 | 2.91 | 3.49 |
| 319 | 0.109 | 0.589 | 1.56 | 2.18 | 2.99 | 3.16 |
| 320 | 0.157 | 0.891 | 2.27 | 2.82 | 3.14 | 3.42 |
| Mean | 0.141 | 1.51 | 3.31 | 4.03 | 4.41 | 4.54 |
| SD | 0.0454 | 0.493 | 1.14 | 1.15 | 1.13 | 1.13 |

The results from all 3 groups show an increase in the concentration of 4-nitroaniline with an increase in zymosan concentration. This increase is greater between 0 and 600 μg zymosan, and gradually plateau out between 600 and 1200 μg zymosan. Mean concentrations of 4-nitroaniline are generally higher in the smokers and COPD patients compared to the healthy volunteers with no obvious difference between the smokers and COPD patients.

### 2. Calibration and Quality Control Samples

The results for the determination of 4-nitroaniline from the analysis of quality control samples are given in Table 10. The mean bias ranged from −3% at 0.100 to 2% at 8.00. Precision ranged from 3.2% at 8.00 μM to 10.6% at 0.100 μM.

The determined concentrations of 4-nitroaniline from the back calculated calibration curves are given in Table 11. The mean bias ranged from −3% at 0.200 to 3% at 10.0. Precision ranged from 2.6% at 2.00 μM to 6.0% at 0.100 μM.

The 4-nitroaniline calibration curve regression parameters are shown in Table 12. The results show a mean slope value of 0.289 with a precision of 3.7%. The range of the R-squared value was 0.9914-0.9995.

TABLE 10
Determined concentrations of 4-nitroaniline in human plasma
for the quality control samples.

| | Curve | Low | Mid | High |
|---|---|---|---|---|
| | Number | (0.100 μM) | (2.00 μM) | (8.00 μM) |
| | 1 | 0.101 | 1.85 | 7.61 |
| | | 0.100 | 1.97 | 7.86 |
| | 2 | 0.0942 | 1.95 | 8.30 |
| | | #0.0836 | 1.99 | 8.31 |
| | 3 | 0.0851 | 1.88 | 7.65 |
| | | 0.0992 | 1.93 | 7.79 |
| | 4 | 0.104 | 1.98 | 8.10 |
| | | #0.117 | 2.03 | 8.19 |
| | 5 | 0.0977 | 1.97 | 8.20 |
| | | 0.100 | 1.99 | 7.89 |
| | 6 | 0.107 | 2.01 | 8.10 |
| | | 0.107 | 2.02 | 8.17 |
| | 7 | 0.108 | 2.21 | 8.21 |
| | | 0.0931 | 2.05 | 8.26 |
| | 8 | 0.0915 | 2.00 | 8.47 |
| | | #0.0817 | 2.04 | 8.60 |
| | 9 | #0.0796 | 2.00 | 8.01 |
| | | 0.0913 | 1.98 | 8.25 |
| | 10 | 0.103 | 2.10 | 8.34 |
| | | 0.112 | 2.15 | 8.56 |
| | 11 | 0.0974 | 2.07 | 8.24 |
| | | 0.0892 | 2.08 | 8.49 |
| | 12 | 0.102 | 2.06 | 8.28 |
| | | #0.0790 | 2.04 | 8.14 |
| Mean | | 0.0968 | 2.01 | 8.17 |
| S.D. | | 0.0103 | 0.0782 | 0.261 |
| Precision | | 10.6 | 3.9 | 3.2 |
| % Bias | | −3 | 1 | 2 |
| n | | 24 | 24 | 24 |
| Overall Precision | | 5.9 | | |
| #> 15% Accuracy | | | | |

TABLE 11
Determined concentrations of 4-nitroaniline in human plasma from the back calculated calibration curves.

| | Analytical Run Number | 0.0500 | 0.100 | 0.200 | 0.500 | 1.00 | 2.00 | 5.00 | 10.0 |
|---|---|---|---|---|---|---|---|---|---|
| | 1 | 0.0500 | 0.0985 | 0.207 | 0.492 | 1.02 | 1.99 | 4.91 | 10.0 |
| | 2 | 0.0518 | 0.0991 | 0.178 | 0.481 | 1.01 | 2.01 | 5.16 | 10.8 |
| | 3 | 0.0487 | 0.107 | 0.189 | 0.523 | 1.03 | 1.97 | 4.68 | 10.1 |
| | 4 | 0.0478 | 0.106 | 0.215 | 0.484 | 0.969 | 2.00 | 4.88 | 9.93 |
| | 5 | 0.0487 | 0.106 | 0.199 | 0.477 | 0.986 | 2.00 | 5.09 | 10.1 |
| | 6 | 0.0499 | 0.0995 | 0.203 | 0.510 | 0.945 | 2.08 | 4.98 | 9.89 |
| | 7 | 0.0519 | 0.0933 | 0.193 | 0.527 | 0.959 | 1.99 | 5.14 | 10.3 |
| | 8 | 0.0542 | 0.0889 | 0.178 | 0.493 | 0.978 | 2.14 | 5.11 | 10.8 |
| | 9 | 0.0512 | 0.0948 | 0.202 | 0.503 | 0.973 | 1.98 | 5.11 | 10.3 |
| | 10 | 0.0509 | 0.0971 | 0.197 | 0.494 | 0.982 | 2.04 | 5.09 | 10.1 |
| | 11 | 0.0524 | 0.0930 | 0.191 | 0.493 | 0.947 | 2.05 | 5.31 | 10.4 |
| | 12 | 0.0524 | 0.0929 | 0.191 | 0.495 | 1.00 | 1.95 | 5.11 | 10.8 |
| Mean | | 0.0508 | 0.0980 | 0.195 | 0.498 | 0.983 | 2.02 | 5.05 | 10.3 |
| S.D. | | 0.00187 | 0.00587 | 0.0110 | 0.0156 | 0.0274 | 0.0530 | 0.163 | 0.340 |
| Precision | | 3.7 | 6.0 | 5.6 | 3.1 | 2.8 | 2.6 | 3.2 | 3.3 |
| % Bias | | 2 | −2 | −3 | 0 | −2 | 1 | 1 | 3 |
| n | | 12 | 12 | 12 | 12 | 12 | 12 | 12 | 12 |

TABLE 12
Calibration curve regression parameters.

| | Curve | | | R- | | | Regression |
|---|---|---|---|---|---|---|---|
| | Number | Slope | Intercept | Squared | LOQ | ULQ | Footnote(s) |
| | 1 | 0.306731 | −0.00299035 | 0.9995 | 0.0500 | 10.0 | 1 |
| | 2 | 0.293280 | 0.00191347 | 0.9957 | 0.0500 | 10.0 | 1 |
| | 3 | 0.306437 | −0.00437884 | 0.9969 | 0.0500 | 10.0 | 1 |
| | 4 | 0.295852 | −0.00432591 | 0.9973 | 0.0500 | 10.0 | 1 |
| | 5 | 0.291634 | −0.00595502 | 0.9985 | 0.0500 | 10.0 | 1 |
| | 6 | 0.287558 | −0.00443996 | 0.9989 | 0.0500 | 10.0 | 1 |
| | 7 | 0.287984 | −0.00180215 | 0.9974 | 0.0500 | 10.0 | 1 |
| | 8 | 0.274692 | 0.000159312 | 0.9914 | 0.0500 | 10.0 | 1 |
| | 9 | 0.285498 | −0.00318718 | 0.9989 | 0.0500 | 10.0 | 1 |
| | 10 | 0.275490 | −0.00268306 | 0.9995 | 0.0500 | 10.0 | 1 |
| | 11 | 0.278743 | −0.00108092 | 0.9963 | 0.0500 | 10.0 | 1 |
| | 12 | 0.281890 | −0.00192762 | 0.9968 | 0.0500 | 10.0 | 1 |
| Mean | | 0.288816 | −0.00255819 | 0.9973 | | | |
| S.D. | | 0.0106541 | 0.00218776 | 0.0022 | | | |
| Precision | | 3.7 | −85.5 | 0.2 | | | |
| n | | 12 | 12 | 12 | | | |

Regression Footnote(s):
1) Resp. = Slope * Conc. + Intercept

### Summary

Human plasma samples were analysed for 4-nitroaniline using a validated LC-MS analytical method. The data obtained for quality control samples analysed with the test samples were within acceptable limits and give confidence in the data generated in the study.

The results from all 3 groups show an increase in the concentration of 4-nitroaniline with an increase in zymosan concentration. This increase is greater between 0 and 600 μg zymosan, and gradually plateau out between 600 and 1200 μg zymosan. Mean concentrations of 4-nitroaniline are generally higher in the smokers and COPD patients compared to the healthy volunteers with no obvious difference between the smokers and COPD patients.

### Example 4

### Protease Activity in Synovial Fluid

Synovial fluid (SF) was obtained from patients with rheumatoid arthritis (7 subjects) and systemic lupus erythematosus (SLE) (1 subject).

### Sample Preparation

To get an estimate of expected activity range, the samples were assayed for total MMP protein content with commercial ELISA kit. MMP1 levels ranged from 3.5-10 nM, MMP9 0.05-0.81 nM. Total protein levels were 30-56 mg/ml (Biorad DC protein assay kit, reagent A, B and S).

The amount of each sample was adjusted so that approximately 4 mg of total protein was used in the assay (Table 13).

The samples were made up to 180 μl with a buffer consisting of 50 mM Tricine pH 7.5, 200 mM NaCl, 10 mM CaCl₂, 20 μM ZnCl₂ and 0.05% Brij-35.

### MMP Activity Assay

To the samples was added 20 μl of the substrate Methyl 1-acetyl-L-prolyl-L-leucylglycyl-α-R-(4-nitrophenylamino)-glycyl-L-leucylglycinate (Example 1.2) solution (10 mM in methanol). After 3 h incubation at 37° C., the reaction was stopped with 5 μl 200 mM EDTA. The samples were filtered through a precolumn (Waters OASIS HLB Icc, part no. 94225, pre-conditioned with 1 ml of ethanol followed by 1 ml water) which was eluted with 1 ml water and 1 ml of 90% ethanol. The samples were evaporated to dryness under a stream of nitrogen, redissolved in 200 μl 50% ethanol and vigorously mixed with vortex mixer and centrifuged (1000 g, 2 min). Of the supernatant, 10-160 μl aliquots were analysed for the reporter 4-nitroaniline with HPLC (detection as in example 3 or with UV at 380 nM). A standard curve was obtained by from samples spiked with quantified amounts of the reporter (4NA) and treated in the same way as the analytical samples.

TABLE 13
MMP activity in synovial fluid.

| | MMP1 | MMP9 | Protein | Sample | 4NA |
|---|---|---|---|---|---|
| Subject | (nM) | (nM) | (mg/ml) | (μl) | (nmol/ml SF) |
| RA238 | 5.8 | 0.37 | 44.2 | 92.3 | 26.4 |
| RA240 | 10.4 | 0.07 | 48.6 | 84.0 | 16.5 |
| RA247 | 7.5 | 0.07 | 56.5 | 72.2 | 20.6 |
| RA248 | 6.9 | 0.07 | 30.0 | 136 | 12.0 |
| RA249 | 3.5 | 0.81 | 50.6 | 80.6 | 94.4 |
| RA250 | 3.5 | 0.46 | 48.6 | 84.0 | 17.4 |
| RA251 | 4.5 | 0.54 | 39.8 | 102.5 | 15.9 |
| SLE524 | 4.7 | 0.31 | 44.9 | 90.9 | 23.9 |

Similarly, using pooled sample of synovial fluid from all subjects in Table 13, Methyl 1-acetyl-L-prolyl-L-leucylglycyl-α-S-(biphenyl-4-ylmethoxy)-glycyl-L-leucylglycinate (Example 1.3) was used following the same procedure, but analysing for the reporter 4-biphenylmethanol. With this substrate, the release was 33.2 nmol/ml SF.

All publications mentioned in the above specification are herein incorporated by reference. Various modifications and variations to the present invention will be apparent to those skilled in the art without departing from the scope and spirit of the present invention. Although the present invention has been described in connection with specific preferred embodiments, it should be understood that the invention as claimed should not be unduly limited to such specific embodiments.

1. A method for determining the activity of a protease in a sample comprising the steps of:
(i) admixing said sample with a substrate, wherein the substrate has the formula (1a) 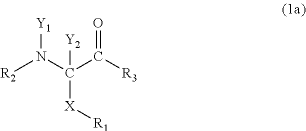
wherein:
R₁ is a hydrocarbyl group
R₂ is a first peptide moiety
R₃ is a second peptide moiety and
X is selected from the group consisting of O, S and NH;
Y₁ is a suitable substituent;
Y₂ is a suitable substituent;
and
(ii) determining the activity of said protease by detecting the presence of a reporter having the formula H—X—R₁, wherein:
X is selected from the group consisting of O, S and NH; and
R₁ is a hydrocarbyl group.

2. A method for determining the efficacy of a protease-modulator comprising the steps of contacting a protease with said protease-modulator and determining the activity of said protease by the method according to claim 1.

3. The method according to claim 2 wherein said protease-modulator is a protease-inhibitor.

4. The method according to claim 2 wherein said protease-modulator is a protease-activator.

5. The method according to claim 4 wherein said protease-activator is zymosan.

6. A method for determining the efficacy of a candidate protease-modulator comprising the steps of contacting a protease with said candidate protease-modulator and determining the activity of said protease by the method according to claim 1.

7. The method according to claim 6 wherein said candidate protease-modulator is a candidate protease-inhibitor.

8. The method according to claim 6 wherein said candidate protease-modulator is a candidate protease-activator.

9. The method according to claim 1 wherein said sample is selected from the group consisting of: urine, whole blood, blood plasma, blood serum, synovial fluid, saliva, sputum, bronchoalveolar fluids, cerebrospinal fluid, nasal lavage, lung lining fluid, tear fluid and skin blister fluid.

10. The method according to claim 1 wherein said sample is selected from the group consisting of: a cell culture, tissue, tissue slices and homogenised tissue.

11. The method according to claim 1 wherein said protease is a matrix metalloproteinase (MMP) EC 3.4.24-.

12. The method according to claim 11 wherein said matrix metalloproteinase is selected from the group consisting of MMP8 (EC 3.4.24.34), MMP9 (EC 3.4.24.35), MMP12 (EC3.4.24.65) and MMP13 (EC3.4.24.-).

13. The method according to claim 12 wherein said matrix metalloproteinase is MMP9 (EC 3.4.24.35).

14. The method according to any claim 1 wherein the hydrocarbyl group R₁ is selected from the group consisting of an aryl, heteroaryl, aryloxyaryl, biaryl, alkyl, cycloalkyl, heterocycloalkyl group and derivatives thereof.

15. The method according to claim 14 wherein R₁ is selected from the group consisting of an aryl, heteroaryl, aryloxyaryl, biaryl and derivatives thereof.

16. The method according to claim 15 wherein R₁ is selected from the group consisting of: 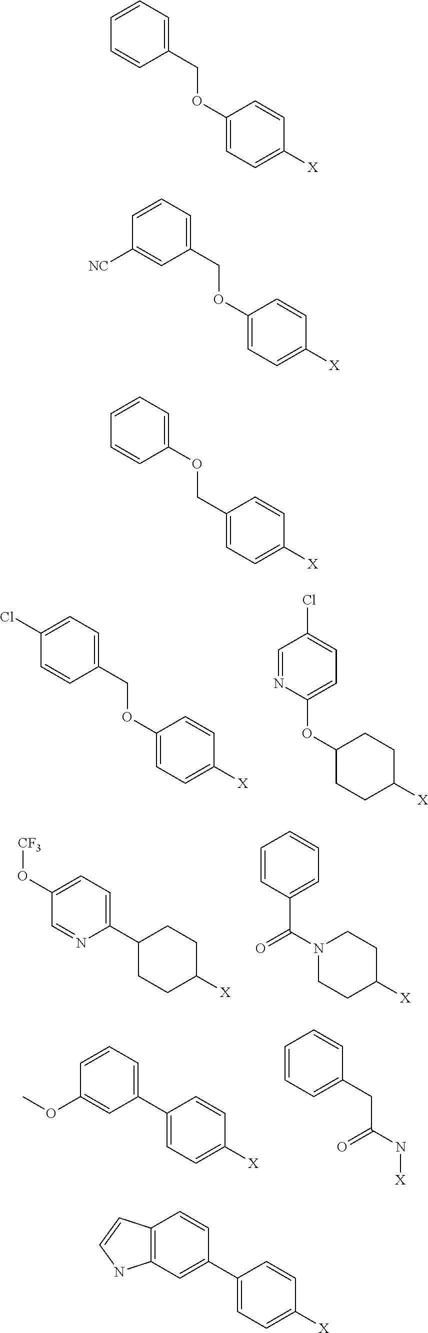 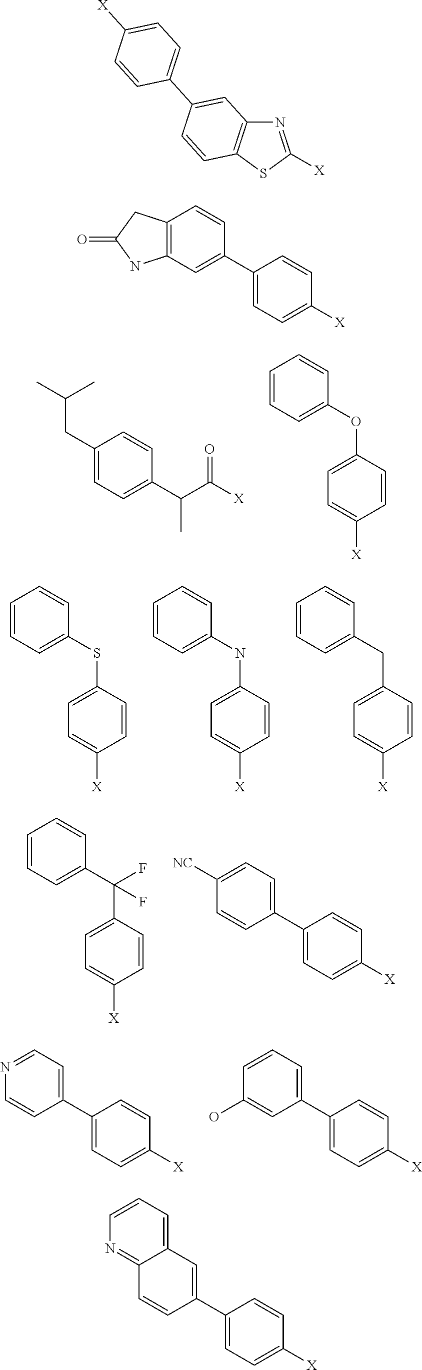 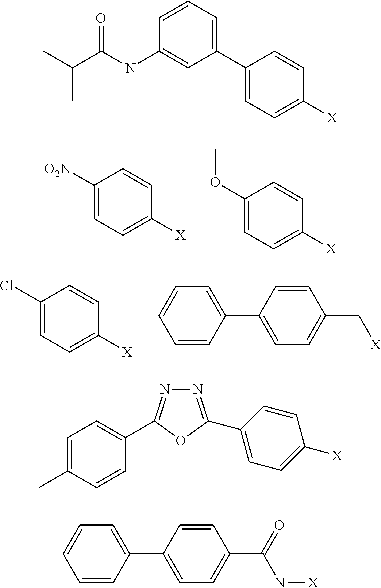
wherein X denotes the remainder of the substrate of formula (1a) and/or the remainder of the reporter having the formula H—X—R₁.

17. The method according to claim 1 wherein said substrate is methyl 1-acetyl-L-prolyl-L-leucylglycyl-α-R-(4-nitrophenylamino)-glycyl-L-leucyl-β-alaninate.

18. The method according to claim 17 wherein said reporter is 4-nitroaniline.

19. A substrate having the formula (1a) 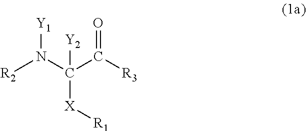
wherein:
R₁ is a hydrocarbyl group
R₂ is a first peptide moiety
R₃ is a second peptide moiety and
X is selected from the group consisting of O, S and NH;
Y₁ is a suitable substituent;
Y₂ is a suitable substituent.

20. A substrate capable of being cleaved at a peptide bond between a carbonyl group and a —NH—CH(X—R₁)-group by a protease;
wherein:
R₁ is a hydrocarbyl group and
X is selected from the group consisting of O, S and NH.

21. A reporter having the formula H—X—R₁,
wherein:
R₁ is a hydrocarbyl group and
X is selected from the group consisting of O, S and NH;
and wherein said reporter is derived from a substrate of the formula (1a) as defined in claim 1;
but wherein said reporter is not 2-(4-Isobutyl-phenyl)-propionic acid.

22. The reporter according to claim 21 wherein the hydrocarbyl group R₁ is selected from the group consisting of an aryl, heteroaryl, aryloxyaryl, biaryl, alkyl, cycloalkyl, heterocycloalkyl group and derivatives thereof.

23. The reporter according to claim 22 wherein R₁ is selected from the group consisting of an aryl, heteroaryl, aryloxyaryl, biaryl and derivatives thereof.

24. The reporter according to claim 23 wherein R₁ is selected from the group consisting of: 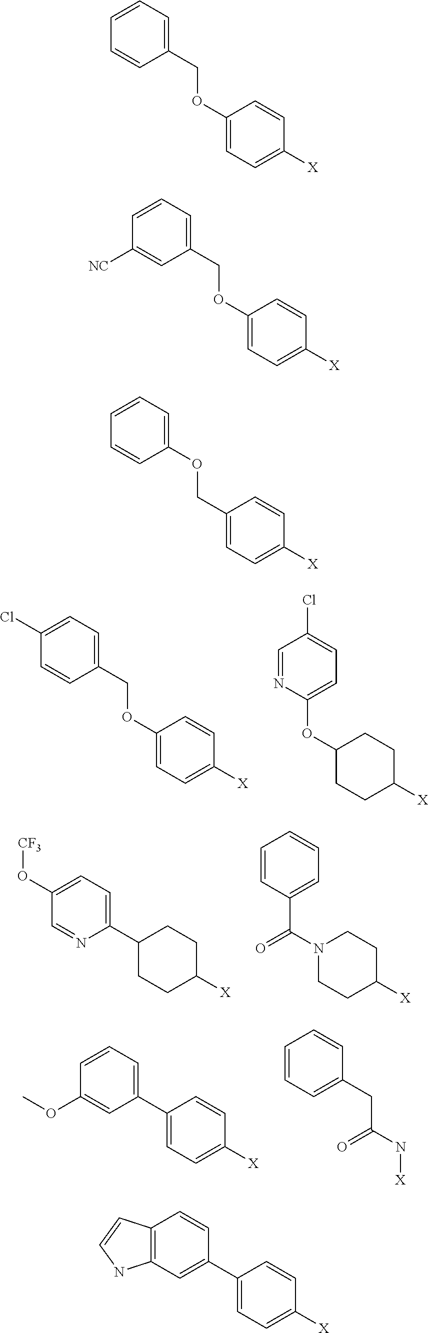 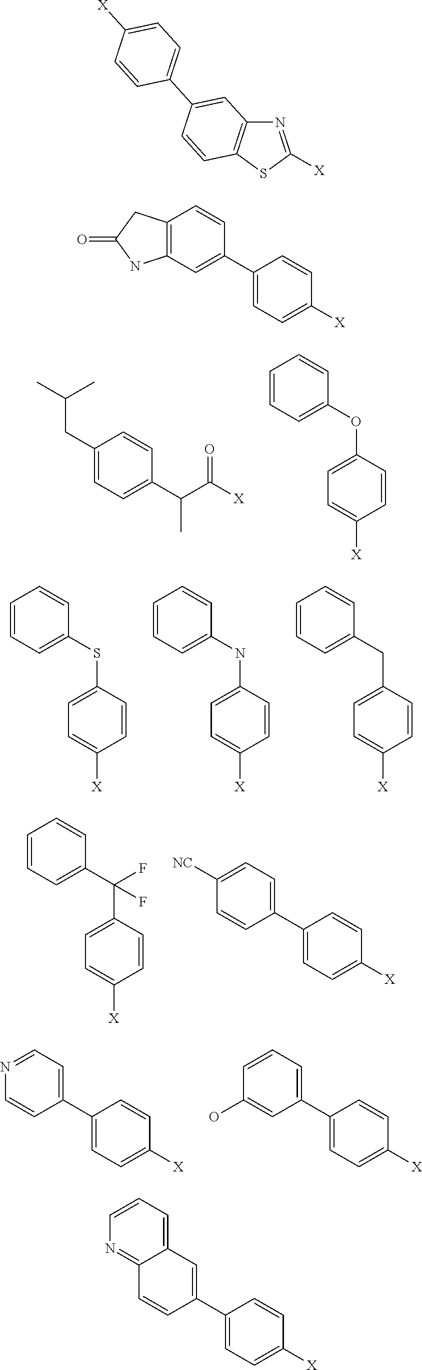 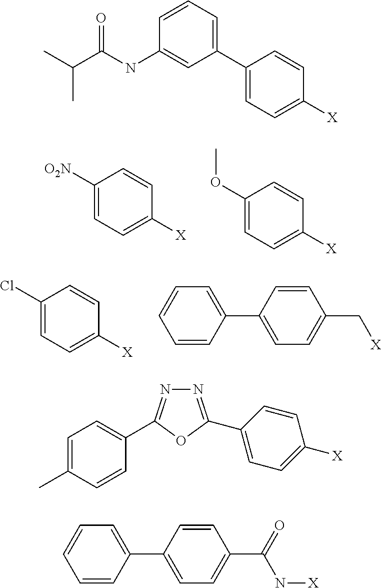
wherein X denotes the remainder of the reporter.

25. A kit for determining the activity of a protease in a sample comprising:
(i) a substrate as defined in claim 1; and
(ii) means for detecting a reporter as defined in claim 1 in a sample.

26-27. (canceled)

28. A method for diagnosing a disease or disorder in a subject comprising the steps of obtaining a sample from said subject and determining the activity of a protease in said sample by the method according to claim 1.

29-30. (canceled)

31. The method according to claim 28, wherein said disease is chronic obstructive pulmonary disease (COPD).

32. A method for determining the efficacy of a protease-modulator; wherein said method comprises the steps of:
(i) admixing said protease-modulator with a protease and a substrate having the formula (1a) as defined in claim 1;
(ii) determining the activity of said protease by detecting the presence of a reporter having the formula H—X—R₁, as defined in claim 1.

33. A method for determining the efficacy of a candidate protease-modulator; wherein said method comprises the steps of:
(i) admixing said candidate protease-modulator with a protease and a substrate having the formula (1a) as defined in claim 1; and
(ii) determining the activity of said protease by detecting the presence of a reporter having the formula H—X—R₁, as defined in claim 1.

34. A method for identifying a protease-modulator; wherein said method comprises the steps of:
(i) admixing a candidate protease-modulator with a protease and a substrate having the formula (1a) as defined in claim 1; and
(ii) determining the activity of said protease by detecting the presence of a reporter having the formula H—X—R₁, as defined in claim 1.

35. A process comprising the steps of identifying a protease-modulator, preparing more of an identified protease-modulator and/or then formulating more of the identified protease-modulator; wherein said identification part comprises the steps of:
(i) admixing a candidate protease-modulator with a protease and a substrate having the formula (1a) as defined in claim 1; and
(ii) determining the activity of said protease by detecting the presence of a reporter having the formula H—X—R₁, as defined in claim 1.

36. (canceled)
